SEQUENCE LISTING (27 sequences)

SEQ ID NO: 1 - protein, 315 aa, Artificial Sequence
  Follistatin isoform FST344 precursor [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 2 - protein, 288 aa, Artificial Sequence
  Follistatin isoform FST317 precursor [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 3 - protein, 328 aa, Artificial Sequence
  Follistatin isoform X1 [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCISRYSGLRKEKEKTEAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 4 - protein, 328 aa, Artificial Sequence; identical to SEQ ID NO: 3
  Follistatin isoform X2 [Homo sapiens]

SEQ ID NO: 5 - protein, 314 aa, Artificial Sequence
  Follistatin isoform X3 [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCITKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 6 - protein, 314 aa, Artificial Sequence; identical to SEQ ID NO: 5
  Follistatin isoform X4 [Homo sapiens]

SEQ ID NO: 7 - protein, 308 aa, Artificial Sequence
  Follistatin isoform X5 [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCISRYSGLRKEKEKTEAKSCEDIQCTGGKKCLWDFKVGRGRCSLCDELCPDSKSDEPVCASDNATYASECAMKEAACSSGVLLEVKHSGSCNSPLYSPI

SEQ ID NO: 8 - protein, 301 aa, Artificial Sequence
  Follistatin isoform X6 [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCISRYSGLRKEKEKTEAKSCEDIQCTGGKKCLWDFKVGRGRCSLCDELCPDSKSDEPVCASDNATYASECAMKEAACSSGVLLEVKHSGSCN

SEQ ID NO: 9 - protein, 287 aa, Artificial Sequence
  Follistatin isoform X7 [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCITKSCEDIQCTGGKKCLWDFKVGRGRCSLCDELCPDSKSDEPVCASDNATYASECAMKEAACSSGVLLEVKHSGSCN

SEQ ID NO: 10 - protein, 279 aa, Artificial Sequence
  Follistatin isoform X8 [Homo sapiens]
MIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCISRYSGLRKEKEKTEAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 11 - protein, 111 aa, Artificial Sequence
  Follistatin, isoform CRA_b [Homo sapiens]
GNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKVYQ

SEQ ID NO: 12 - protein, 288 aa, Artificial Sequence
  FST [Homo sapiens]
GNCWLRQAKNGRCQVLYKTELSKEECCSTGRLSTSWTEEDVNDNTLFKWMIFNGGAPNCIPCKETCENVDCGPEKKCRMNKKNKPRCVCAPDCSNITWKGPVCGLDGKTYCNECALLKARCKEQPELEVQYQGRCKKTCRDVFCPGSSTCVVDQTNNAYCVTCNRICPEPASSEQYLCGNDGVTYSSACHLRKATCLLGRSIGLVYEGKCIKAKSCEDIQCTGGKKCLWDFKVGRGRCSLCDELCPDSKSDEPVCASDNATYASECAMKEAACSSGVLLEVKHSGSCN

SEQ ID NO: 13 - protein, 288 aa, Artificial Sequence
  Follistatin like 1 [ Homo sapiens]
EEELRSKSKICANVFCGAGRECAVTEKGEPTCLCIEQCKPHKRPVCGSNGKTYLNHCELHRDACLTGSKIQVDYDGHCKEKKSVSPSASPVVCYQSNRDELRRRIIQWLEAEIIPDGWFSKGSNYSEILDKYFKNFDNGDSRLDSSEFLKFVEQNETAINITTYPDQENNKLLRGLCVDALIELSDENADWKLSFQEFLKCLNPSFNPPEKKCALEDETYADGAETEVDCNRCVCACGNWVCTAMTCDGKNQKGAQTQTEEEMTRYVQELQKHQETAEKTKRVSTKEI

SEQ ID NO: 14 - protein, 24 aa, Artificial Sequence
  Follistatin-N-terminal domains
TCENVDCGPGKKCRMNKKNKPRCV

SEQ ID NO: 15 - protein, 24 aa, Artificial Sequence
  Follistatin-N-terminal domains
TCENVDCGPEKKCRMNKKNKPRCV

SEQ ID NO: 16 - protein, 23 aa, Artificial Sequence
  Follistatin-N-terminal domains
CANVFCGAGRECAVTEKGEPTCL

SEQ ID NO: 17 - protein, 350 aa, Artificial Sequence
  Fetuin A isoform 1
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPADSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 18 - protein, 349 aa, Artificial Sequence
  Fetuin A isoform 2
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 19 - protein, 348 aa, Artificial Sequence
  Fetuin A isoform 3
APHGPGLIYRQPNCDDPETEEAALVAIDYINQNLPWGYKHTLNQIDEVKVWPQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 20 - protein, 321 aa, Artificial Sequence
  Fetuin A isoform 4
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKPVSSQPQPEGANEAVPTPVVDPDAPPSPPLGAPGLPPAGSPPDSHVLLAAPPGHQLHRAHYDLRHTFMGVVSLGSPSGEVSHPRKTRTVVQPSVGAAAGPVVPPCPGRIRHFKV

SEQ ID NO: 21 - protein, 283 aa, Artificial Sequence
  Fetuin A protein isoforms
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPADSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 22 - protein, 40 aa, Artificial Sequence
  Fetuin A protein isoforms linker
LAAPPGHQLHRAHYDLRHTFMGVVSLGSPSGEVSHPRKTR

SEQ ID NO: 23 - protein, 27 aa, Artificial Sequence
  Fetuin A protein isoforms Chain B
TVVQPSVGAAAGPVVPPCPGRIRHFKV

SEQ ID NO: 24 - protein, 282 aa, Artificial Sequence
  Fetuin A protein isoforms Chain A
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 25 - protein, 281 aa, Artificial Sequence
  Fetuin A protein isoforms Chain A
APHGPGLIYRQPNCDDPETEEAALVAIDYINQNLPWGYKHTLNQIDEVKVWPQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKQYGFCKATLSEKLGGAEVAVTCMVFQTQPVSSQPQPEGANEAVPTPVVDPDAPPSPPLGAPGLPPAGSPPDSHVL

SEQ ID NO: 26 - protein, 254 aa, Artificial Sequence
  Fetuin A protein isoforms Chain A
APHGPGLIYRQPNCDDPETEEAALVAIDYINQNLPWGYKHTLNQIDEVKVWPQQPSGELFEIEIDTLETTCHVLDPTPVARCSVRQLKEHAVEGDCDFQLLKLDGKFSVVYAKCDSSPDSAEDVRKVCQDCPLLAPLNDTRVVHAAKAALAAFNAQNNGSNFQLEEISRAQLVPLPPSTYVEFTVSGTDCVAKEATEAAKCNLLAEKPVSSQPQPEGANEAVPTPVVDPDAPPSPPLGAPGLPPAGSPPDSHVL

SEQ ID NO: 27 - protein, 19 aa, Artificial Sequence
  TGF-beta binding domain
CDFQLLKLDGKFSVVYAKC

(19) United States
(12) Patent Application Publication
(10) Pub. No.: US 2025/0325629 A1
(43) Pub. Date: Oct. 23, 2025

(54) METHODS OF PREPARATION OF SECRETOMES, AND USES THEREOF

(71) Applicant: Immunis, Inc., Laguna Beach, CA (US)

(72) Inventor: Jessie Silva PAINTER, Laguna Beach, CA (US)

(21) Appl. No.: 18/274,426

(22) PCT Filed: Jan. 26, 2022
(86) PCT No.: PCT/US2022/013834

§ 371 Date: Jul. 26, 2023

Related U.S. Application Data
(60) Provisional application No. 63/142,456, filed on Jan. 27, 2021.
(60) Provisional application No. 63/169,203, filed on Mar. 31, 2021.

(51) Int. Cl. A61K 38/17; A61K 31/56; A61K 38/18; A61K 38/19; A61K 38/20; A61K 41/00; A61K 45/06; A61P 21/00; C07K 14/47; C12N 5/00; C12N 5/074

(52) U.S. Cl. CPC A61K 38/1741; A61K 31/56; A61K 38/1841; A61K 38/191; A61K 38/204; A61K 41/00; A61K 45/06; A61P 21/00; C07K 14/473; C12N 5/0081; C12N 5/0696; C12N 2500/34; C12N 2500/95

(57) ABSTRACT

Pharmaceutical compositions and methods of producing them are described. The pharmaceutical composition may include (A) a follistatin protein or a variant thereof, and (B) a fetuin A protein or a fragment or variant thereof, wherein (B) is capable of binding to TGF-β, and wherein the total amount of (A) and (B) is greater than 10% of total proteins in the composition. Methods of preparing a secretome composition may include culturing stem cells in a protein-free media comprising trehalose, harvesting supernatant from the sample and replacing the supernatant with a fresh protein-free media comprising trehalose, and combining the harvested supernatant to prepare the secretome composition.

### CROSS-REFERENCE TO RELATED APPLICATIONS

This application is a U.S. National Stage Application under U.S.C. § 371 of International Application No. PCT/US2022/013834, filed Jan. 26, 2022, which claims the benefit of priority under 35 U.S.C. § 119 (e) from U.S. Provisional Application Ser. No. 63/142,456, filed Jan. 27, 2021, U.S. Provisional Application Ser. No. 63/169,203, filed Mar. 31, 2021, and U.S. Provisional Application Ser. No. 63/177,346, filed Apr. 20, 2021, all of which are hereby expressly incorporated by reference in their entireties for all purposes.

### SEQUENCE LISTING

The instant application contains a Sequence Listing which has been submitted electronically in ASCII format and is hereby incorporated by reference in its entirety. Said ASCII copy, created on Jul. 25, 2023, is named IMBIO_002_WO_ST25_Sequence_Listing.txt and is 56.7 KB in size.

### FIELD

The subject matter described herein relates generally to a methods of preparing secretomes and their use for treatment of various conditions, including conditions related to aging.

### BACKGROUND

Periods of muscle disuse often occur in aging as a consequence of illness, injury or recovery from surgery (1-3). These disuse events often lead to the rapid atrophy of skeletal muscle mass and weakness which is associated with poor prognosis and increased risk of re-hospitalization or further injury (4). Age-related loss of muscle mass and strength (5) result in decreased mobility and quality of life while also increasing the risk of other co-morbidities and mortality (6-8). Furthermore, recovery from disuse atrophy is often compromised in aged muscle, never fully achieving the previous baseline muscle size and functional quality (9, 10). Therefore, novel therapies are needed that target aging skeletal muscle and are fully effective to mitigate muscle atrophy while improving muscle recovery following disuse.

A common studied mechanism linked to muscle dysfunction during aging is fibrosis, or the accumulation of extracellular matrix (particularly collagen) in skeletal muscle (11, 12). Excessive muscle collagen deposition impairs the functional capacity to generate force (6, 7, 9, 13). Recent evidence has demonstrated that the abundance of collagen in aged mouse muscle is nearly double compared to their younger counterparts (14) though it is not clear if this is a function of increased collagen expression or poor turnover (15). Muscle disuse in rodents further contributes to the accumulation of collagen (16, 17) thus raising concern that excessive fibrosis may participate in the less than optimal muscle recovery in aging. Concomitant with age-related muscle fibrosis is a decline in the muscle satellite cell pool which is critical to the ability of muscle to recover from injury (18, 19). Moreover, aging is associated with a decline in the number and activity of myogenic satellite cells during injury which are necessary for continued myogenesis (19-21). Therefore, it is reasonable to consider that altered satellite cell function and collagen deposition likely partly contribute to poor quality of aging muscle during disuse and recovery.

While many studies have examined mechanisms of muscle fibrosis and satellite cell function in aging there are still significant gaps in our understanding of what drives aging muscle toward this phenotype and worsened during disuse atrophy and recovery conditions. One possibility is a dysfunctional immune system associated with aging (22, 23); regeneration or regrowth of skeletal muscle during recovery from injury or disuse atrophy require carefully timed and coordinated events of immune cells (e.g., neutrophils, macrophages) that promote a tightly controlled pro and anti-inflammatory local environment to ensure activation and proliferation of satellite cells and proper ECM remodeling (24). Our laboratory and others have highlighted that aged skeletal muscle is present with an impaired pro-inflammatory macrophage response during recovery during disuse atrophy (2, 25). Depletion of macrophage activity (chemical or genetic approaches) during recovery from injury or disuse atrophy leads to muscle fibrosis and incomplete muscle recovery (26-30) thus emphasizing the importance of macrophage function for proper regrowth of muscle.

Few studies have employed interventions during disuse atrophy and recovery in aged skeletal muscle, with the outcome ambivalent (13, 31-33). Though not fully effective on muscle size and function, these studies have highlighted a variety of cellular pathways involved in muscle cell growth including activation of satellite cells (13, 31, 32, 34-36). Therefore, a need still exists for a therapy that promotes aged skeletal muscle mass and function during disuse and recovery.

### SUMMARY

To address this problem, the effectiveness of a secretome product (termed STEM) to promote aged skeletal muscle mass and function during disuse and recovery was produced and studied. STEM was found to mitigate skeletal muscle atrophy during disuse and improve regrowth during recovery. STEM also enhanced the aged muscle macrophage and satellite cell phenotype while also reducing muscle fibrosis.

Aged individuals are at risk to experience periods of disuse atrophy and as a result are faced with slow and incomplete muscle recovery. While several therapies have been employed to mitigate muscle mass loss during disuse and improve recovery, few have proven effective at both. Therefore, the purpose of this study was to examine the effectiveness of a uniquely developed secretome product (STEM) on aged skeletal muscle mass and function during disuse and recovery. Aged (22 months) male C57BL/6 were divided into PBS or STEM treatment (n=30). Mice within each treatment were assigned to either ambulatory control (14 days of normal cage ambulation), 14 days of hindlimb unloading (HU), or 14 days of hindlimb unloading followed by 7 days of recovery. Mice were given an intramuscular delivery into the hindlimb muscle of either PBS or STEM every other day for the duration of their respective treatment group. STEM treatment was found to increase soleus muscle mass, fiber CSA (define), and grip strength during Control and Recovery (why capitalized?) while attenuating atrophy and weakness during HU. Muscle pro-(CD68+) and anti-inflammatory (CD163+) macrophages were increased in ambulatory control mice, while pro-inflammatory only were increased during HU and Recovery with STEM administration. Moreover, STEM increased collagen turnover and Pax7+ cells across all treatment groups. As a follow-up to examine the cell autonomous role of STEM on muscle, C2C12 myotubes were given 4% STEM or 4% horse serum media to examine myotube fusion/size and effects on muscle gene networks. STEM-treated C2C12 myotubes were larger and had a higher fusion index and were related to elevated expression of genes associated with defined pathways associated with extracellular matrix remodeling. The results demonstrate that STEM is a unique cocktail that possesses potent anabolic, immunomodulatory, and cytoskeletal remodeling properties that may have translational potential to improve skeletal muscle across a variety of age-related conditions.

Pharmaceutical compositions and methods of producing them are described. The pharmaceutical composition may include (A) a follistatin protein or a variant thereof, and (B) a fetuin A protein or a fragment or variant thereof, wherein (B) is capable of binding to TGF-β, and wherein the total amount of (A) and (B) is greater than 10% of total proteins in the composition. Methods of preparing a secretome composition may include culturing stem cells in a protein-free media comprising trehalose, harvesting supernatant from the sample and replacing the supernatant with a fresh protein-free media comprising trehalose, and combining the harvested supernatant to prepare the secretome composition.

### BRIEF DESCRIPTION OF THE FIGURES

The foregoing summary, as well as the following detailed description of the invention, will be better understood when read in conjunction with the appended drawing. It should be noted that the invention is not limited to the precise embodiment shown in the drawing.

FIG. 1 illustrate the timeline of various experiments.

FIGS. 2A-2F illustrate results from experiments of a secretome on aged skeletal muscle during 14 days of normal cage ambulation.

FIGS. 3A-3F illustrate results from experiments of a secretome on aged skeletal muscle during 14 days of hindlimb unloading.

FIGS. 4A-4F illustrate results from experiments of a secretome on aged skeletal muscle during 7 days of recovery.

FIGS. 5A-5B illustrate the results from experiments of a secretome on C2C12 myotube fusion, size, and RNA sequencing.

FIGS. 6A-C illustrate expressed factors in secretome identified by ELISA.

FIG. 7 illustrates exemplary sequences of follistatin protein and variants thereof.

### DETAILED DESCRIPTION

All references and publications mentioned herein are expressly incorporated by reference in their entirety for all purposes.

Unless defined otherwise, all technical and scientific terms used herein have the same meaning as commonly understood by one of ordinary skill in the art to which this disclosure belongs. It must be noted that as used herein and in the appended claims, the singular forms “a”, “an”, and “the” include plural referents unless the context clearly dictates otherwise. Thus, for example, reference to “a peptide” includes a plurality of peptides.

The term “about” when used before a numerical designation, e.g., temperature, time, amount, and concentration, including range, indicates approximations which may vary by (+) or (−) 10%, 5% or 1%.

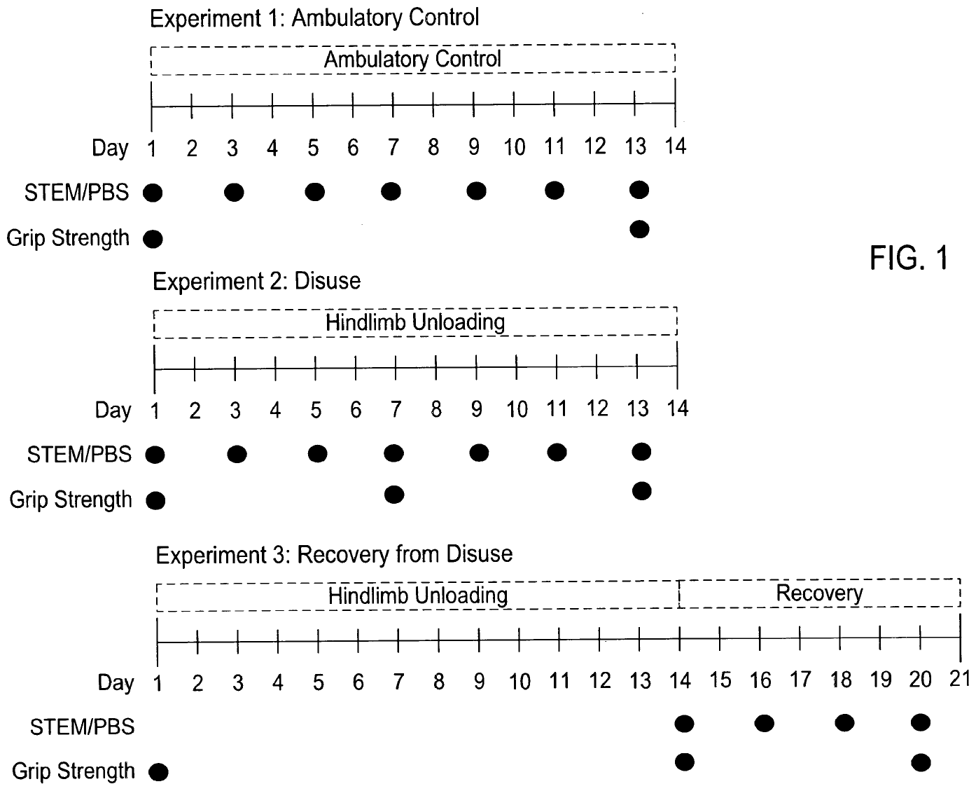

FIG. 1. Experimental Design. Length of each of the three experiments in days. Solid circles indicate time point where intramuscular injection of STEM/PBS and strength measurement occurred.

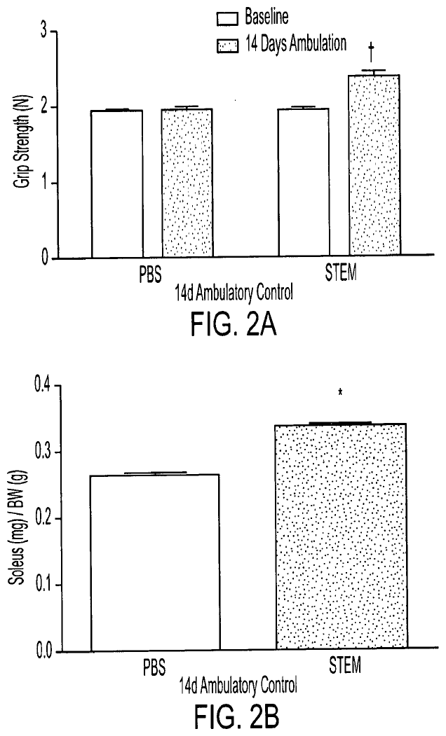

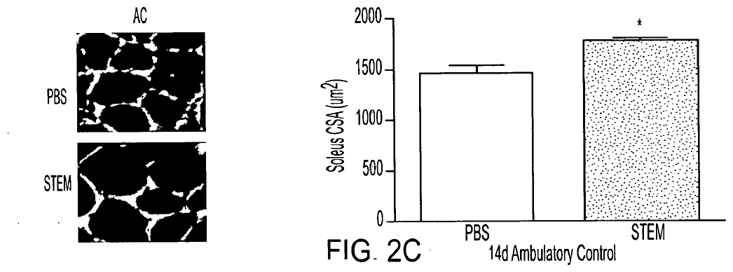

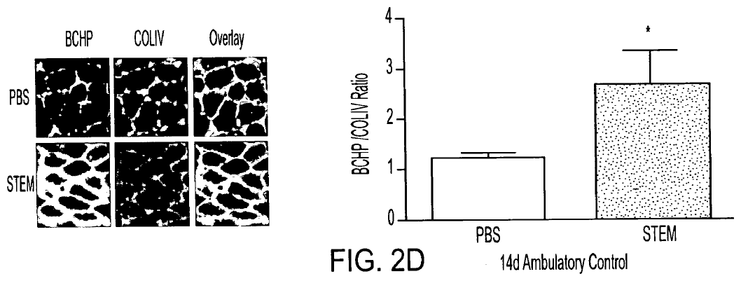

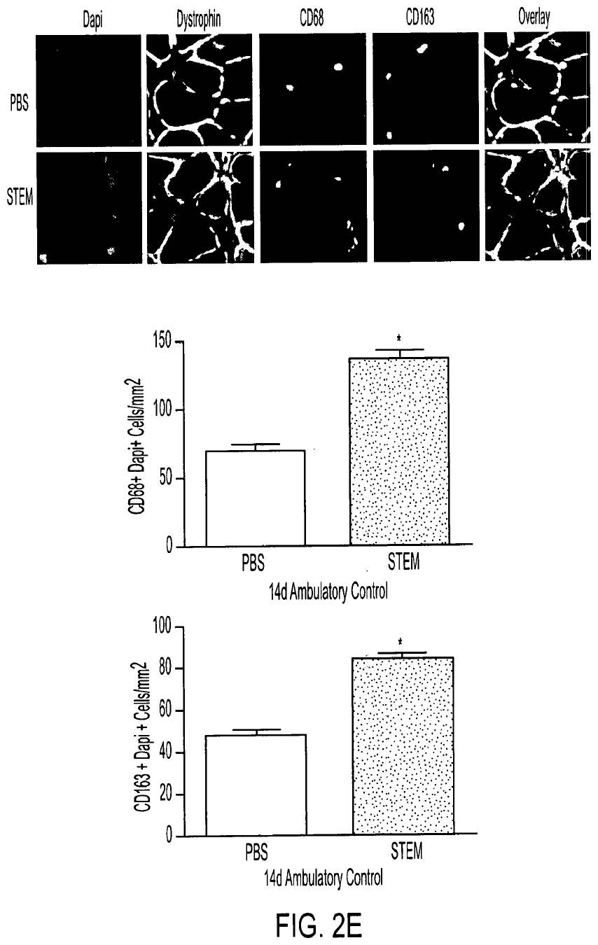

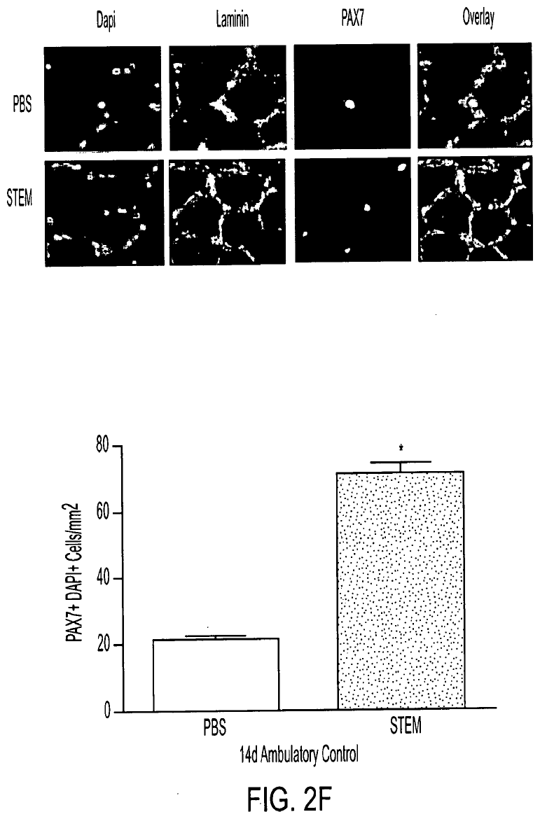

FIGS. 2A-2F. STEM on Aged Skeletal Muscle During 14 days of Normal Cage Ambulation. Panels represent (FIG. 2A) grip strength of PBS and STEM treated mice at Baseline (1 day prior to treatment) and repeated at day 13 of normal cage ambulation (1 day prior to tissue harvest). (FIG. 2B) Soleus muscle mass (mg) of STEM and PBS treated mice and (FIG. 2C) soleus muscle cross sectional area (CSA; um2) after 14 d of normal cage ambulation. (FIG. 2D) Soleus muscle BCHP/COLIV ratio, and (FIG. 2E) CD68+ and CD163+ and (FIG. 2F) PAX7+ abundance in STEM and PBS treated mice after 14 d of normal cage ambulation. Representative immunohistochemistry images are depicted next to each panel. AC denotes ambulatory control. N=5 mice in each group. Results are mean with standard error of the mean, Two-Way ANOVA repeated measures used for (FIG. 2A). T-test between PBS and STEM. †=different to all groups. *=different to PBS.

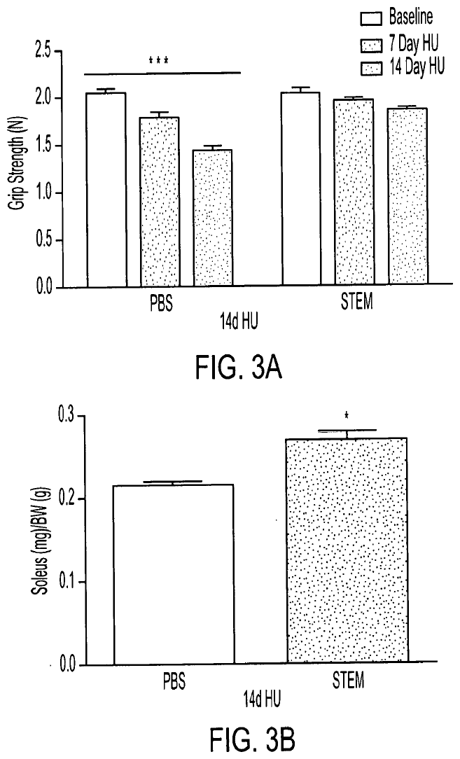

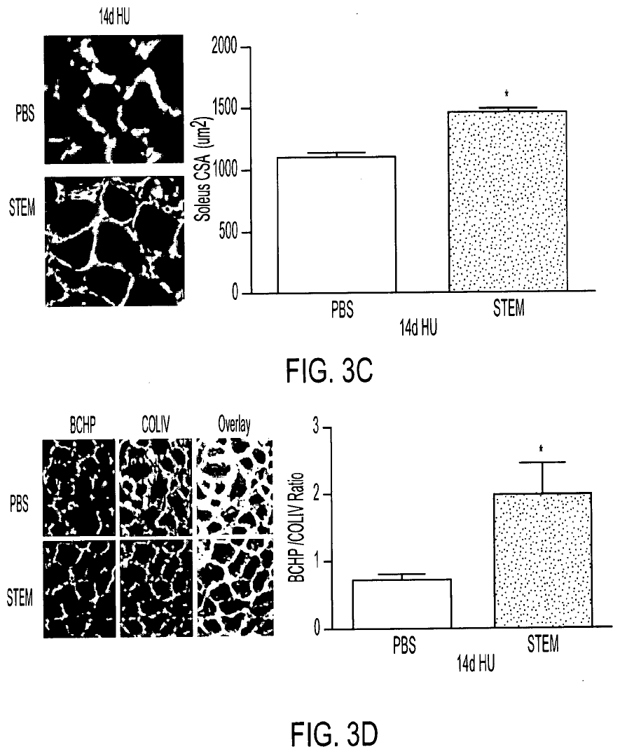

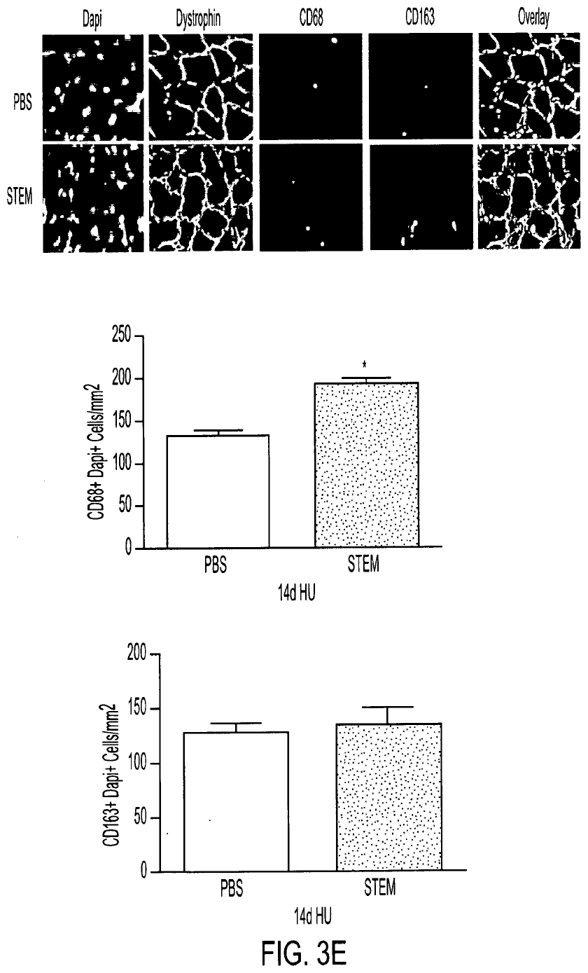

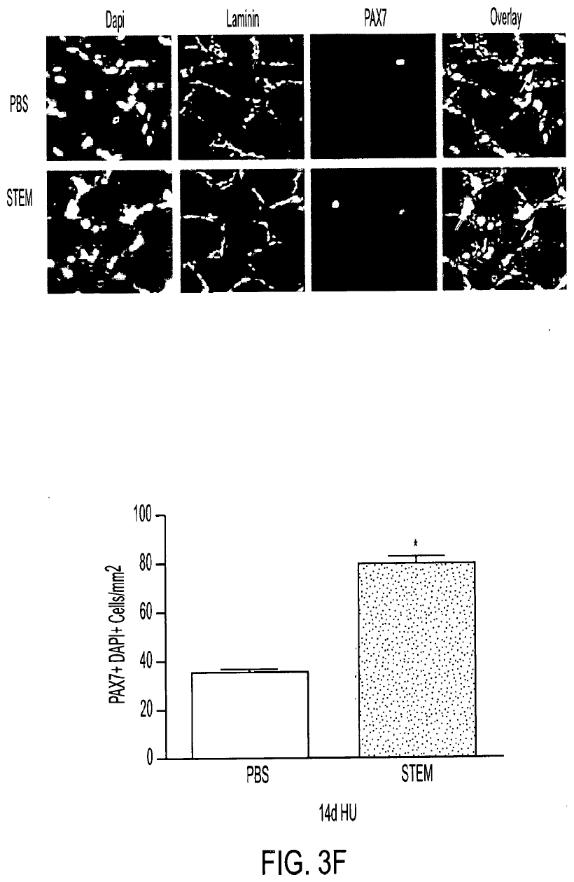

FIGS. 3A-3F. STEM Effects on Aged Skeletal Muscle During 14 days of Hindlimb Unloading. Panels represent (FIG. 3A) grip strength of PBS and STEM treated mice at Baseline (1 day prior to treatment), repeated at day 7 of HU (hindlimb unloading), and day 13 of HU (1 day prior to tissue harvest). (FIG. 3B) Soleus muscle mass (mg) of STEM and PBS treated mice and (FIG. 3C) soleus muscle cross sectional area (CSA; um2) in PBS and STEM treated mice after 14 d of HU. (FIG. 3D) Soleus muscle BCHP/COLIV ratio and (FIG. 3E) CD68+ and CD163+ and (FIG. 3F) PAX7+ abundance in STEM and PBS treated mice after 14 d of HU. N=5 mice in each group. Results are mean with standard error of the mean. T-test between PBS and STEM. ***=all PBS groups different to each other. *=different to PBS.

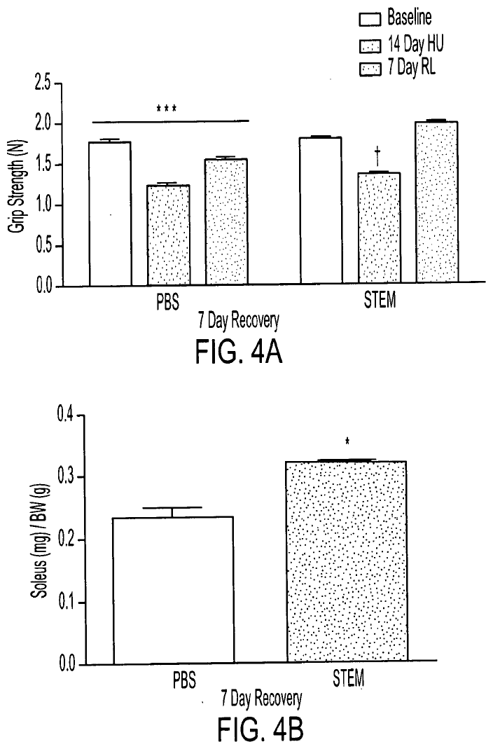

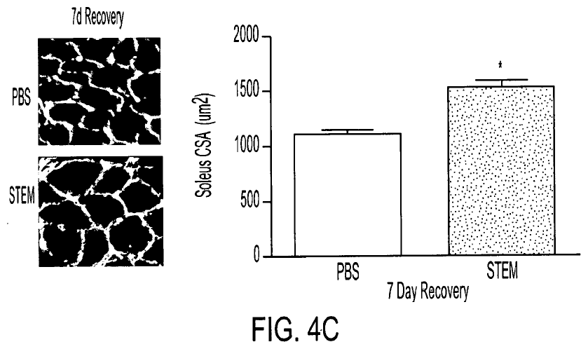

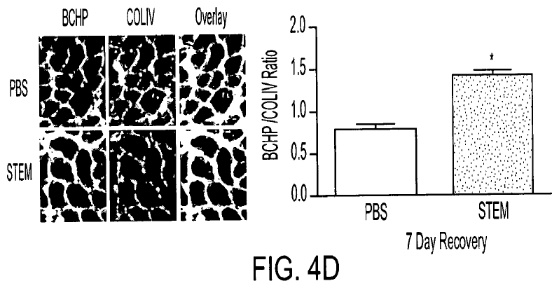

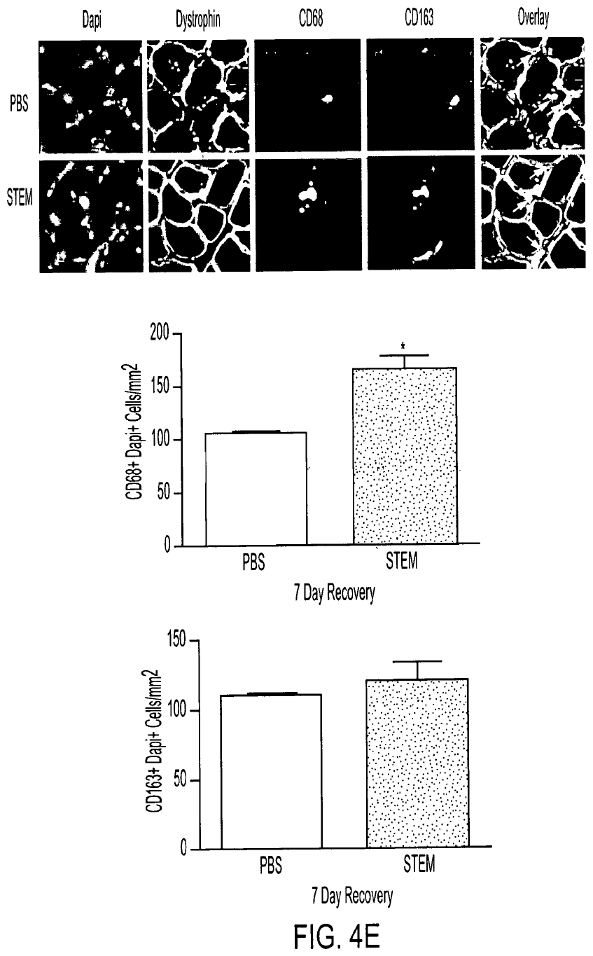

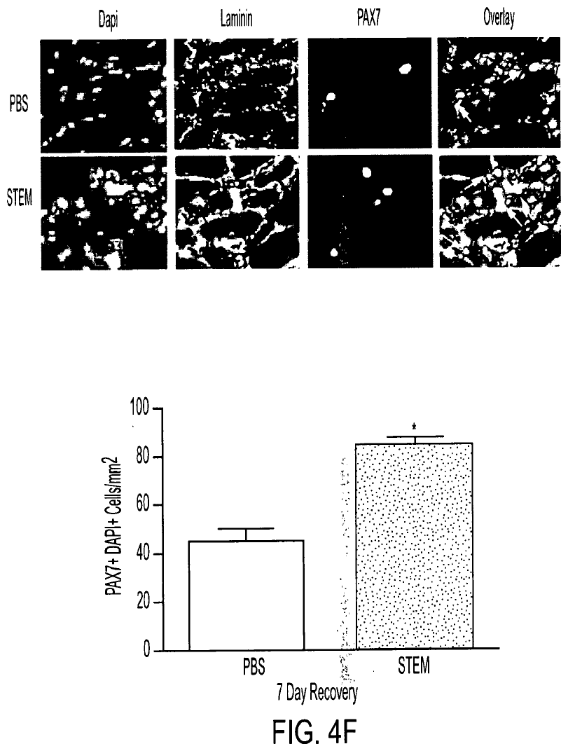

FIGS. 4A-4F. STEM Effects on Aged Skeletal Muscle During 7 Days of Recovery. Panels represent (FIG. 4A) grip strength of PBS and STEM treated mice at Baseline (1 day prior to treatment), repeated at day 14 of HU (hindlimb unloading), and day 6 of RL (Reload, 1 day prior to tissue harvest). (FIG. 4B) Soleus muscle mass (mg) and (FIG. 4C) soleus muscle cross sectional area (CSA; um2) in PBS and STEM treated mice after 7 d of recovery. (FIG. 4D) Soleus muscle BCHP/COLIV and (FIG. 4E) CD68+ and CD163+ and (FIG. 4F) PAX7+ abundance in PBS and STEM treated ambulatory control mice after 7 d of recovery. N=5 mice in each group. Results are mean with standard error of the mean, Two-Way ANOVA repeated measures used for (FIG. 4A). T-test between PBS and STEM. ***=all PBS groups different to each other. †=different to all groups. *=different to PBS.

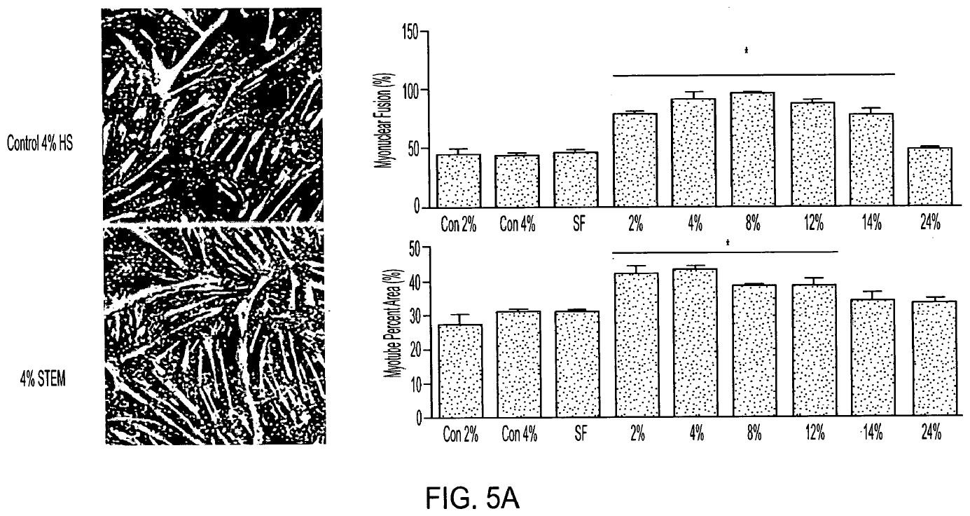

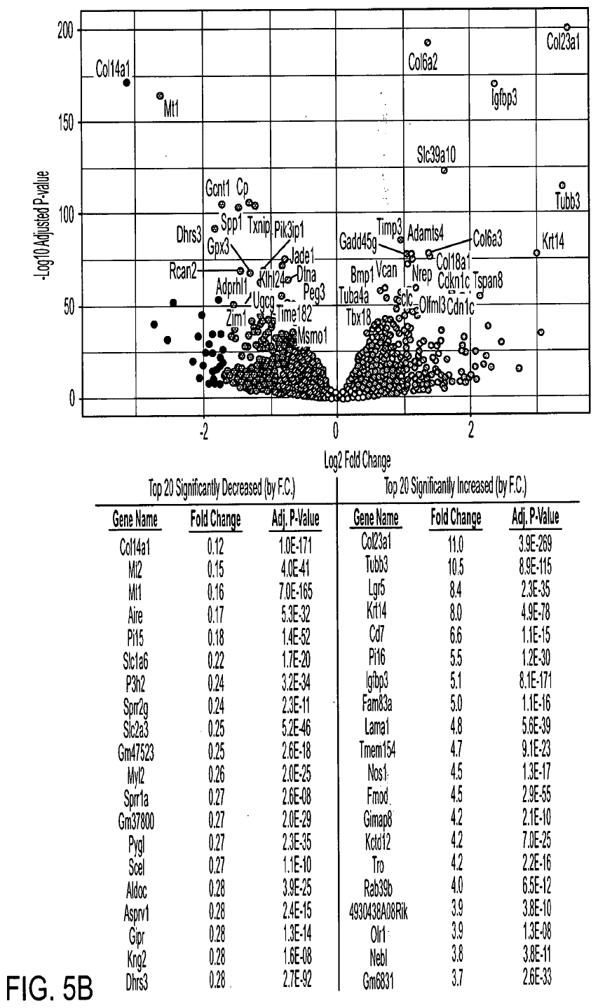

FIGS. 5A-5B. STEM effects C2C12 myotube fusion, size, and RNA sequencing. Panels represent (FIG. 5A) Quantification of controls (2% and 4% horse serum) and 2-24% STEM-treated C2C12 myotubes for myonuclear fusion (%) and myotube size (see Methods). Representative images of 4% horse serum and STEM for myonuclear fusion and myotube percent area. (FIG. 5B) Volcano plot and table of top significantly increased and decreased genes in 4% STEM treated myotubes compared to 4% horse serum (control). Results are mean with standard error of the mean, One-Way ANOVA. *=different to all groups not underneath solid line.

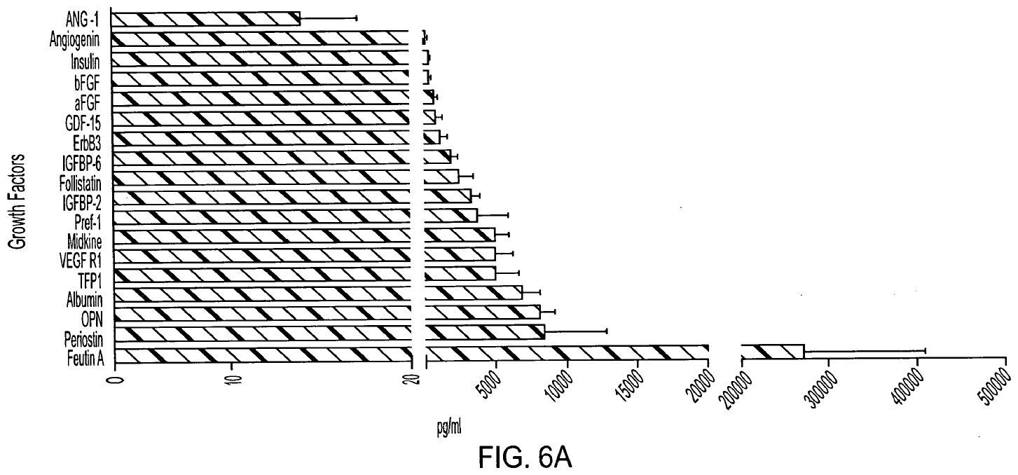

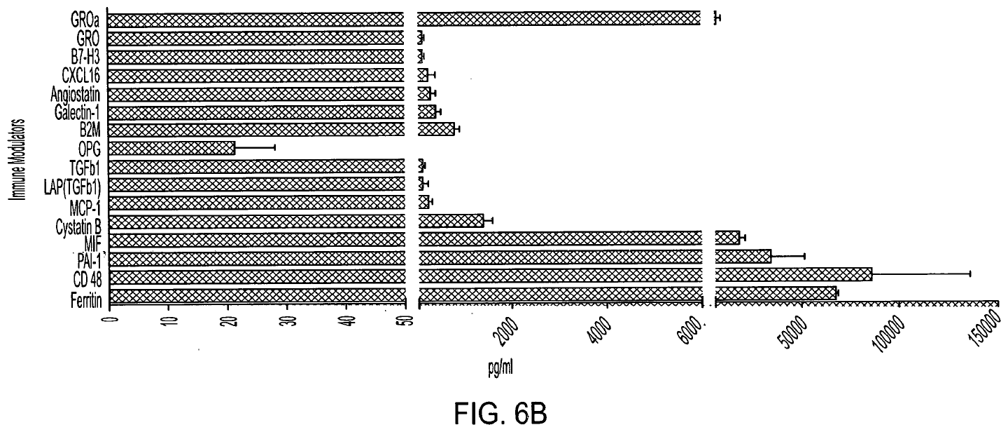

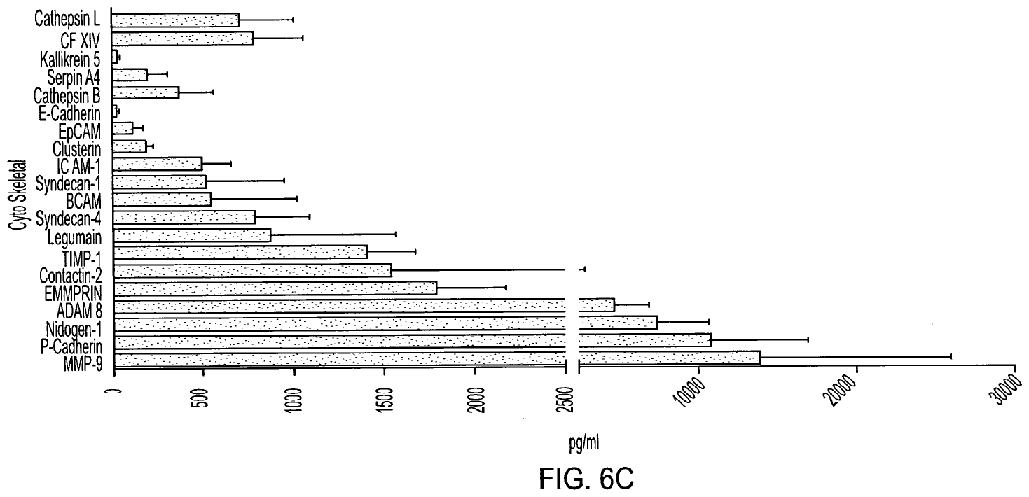

FIGS. 6A-6C. Top Expressed Factors in STEM Identified by Multiplex ELISA. Top identified growth factors (FIG. 6A), immune modulators (FIG. 6B), and cyto-skeletal factors (FIG. 6C). All values are mean with standard error of the mean. Values were obtained via multiplex ELISA from three separate samples and reported in pg/ml. Categorization was assisted using Reactome.

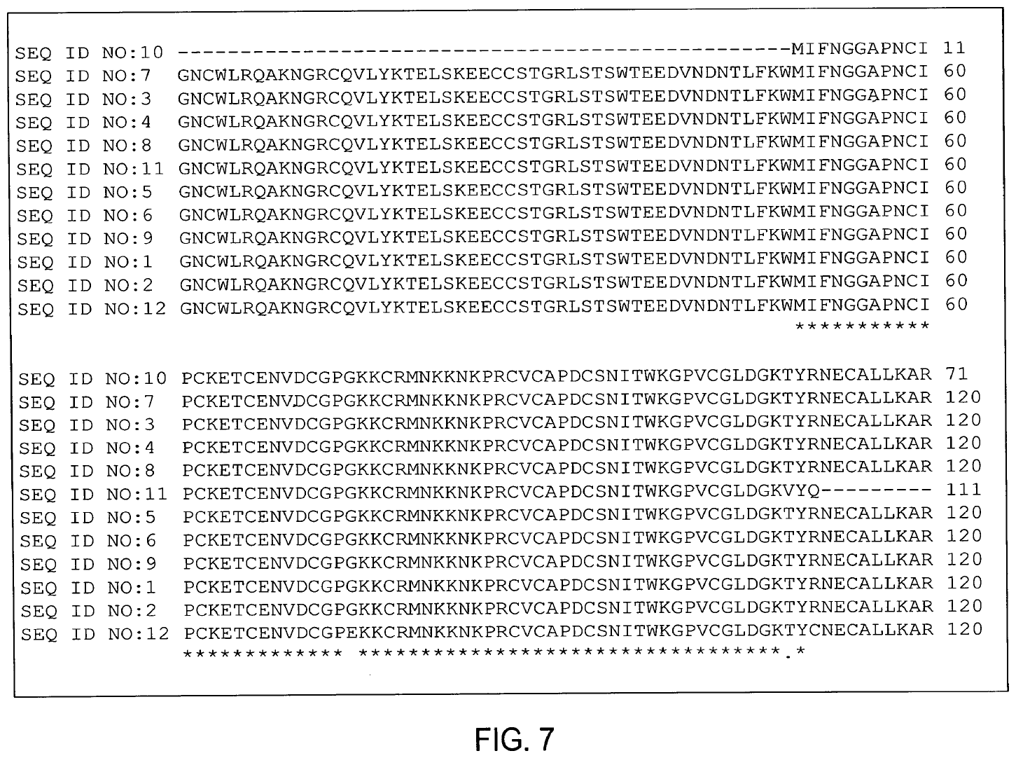

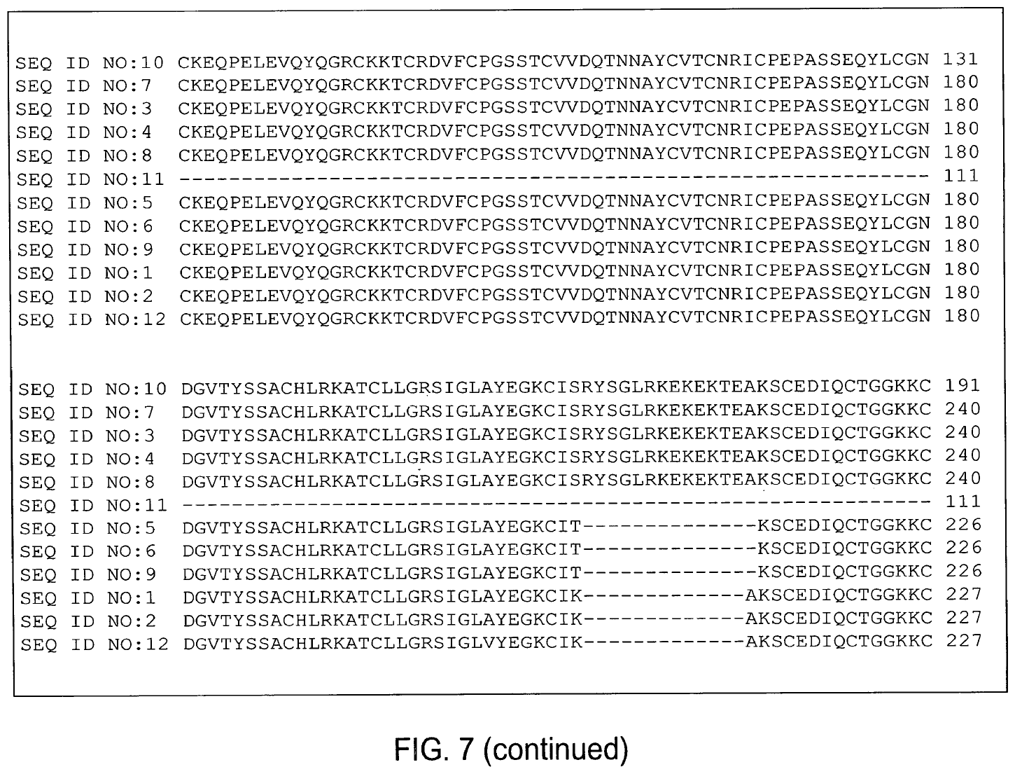

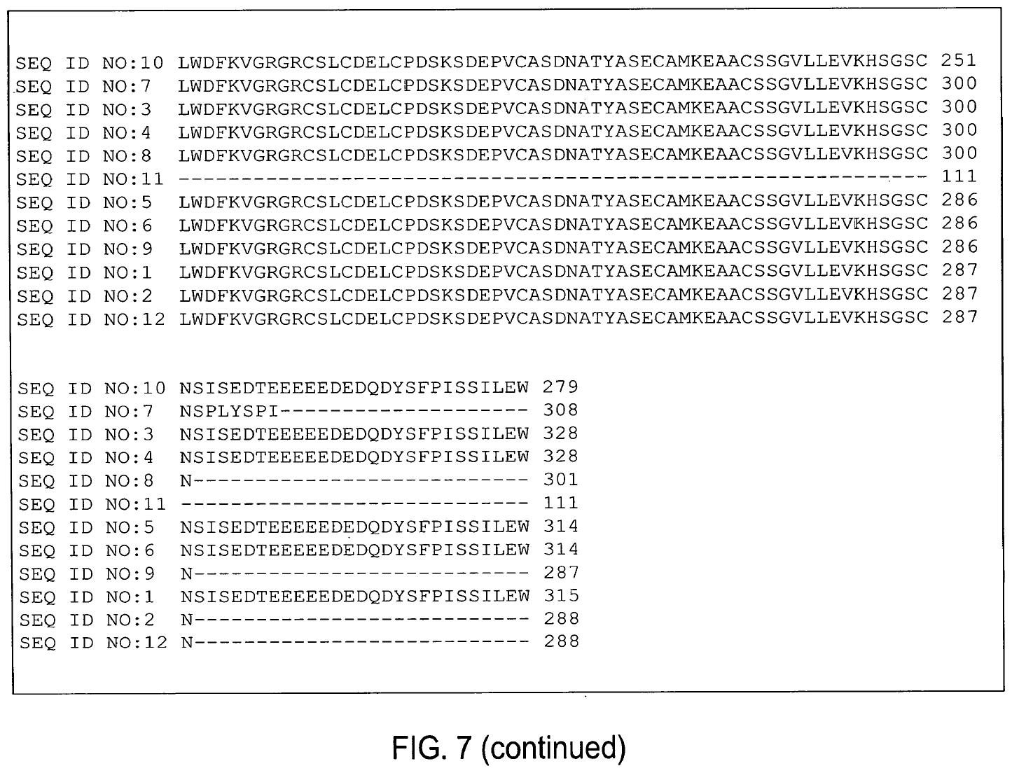

FIG. 7 are exemplary sequences of follistatin protein and variants thereof.

### Materials and Methods

### Animals

Thirty aged male C57BL/6 (B6) at 22 months of age, generously provided by National Institute on Aging mouse colony, were divided into PBS or STEM treatment groups (n=15/group). Mice within each treatment were then assigned to one of 3 experimental groups: a) ambulatory control (14 days of normal cage ambulation) (CON; n=5), b) Disuse as defined as 14 days of hindlimb unloading (HU; n=5), or c) Recovery from Disuse as defined as 14 days of hindlimb unloading followed by 7 days of recovery (Recovery; n=5) (FIG. 1). Animals were housed with ad libitum access to food and water, and maintained on a 12-hour light/dark cycle. All experimental procedures were conducted in accordance with the guidelines set by the Institutional Animal Care and Use Committee (IACUC) at the University of Utah.

### Ambulatory Controls and Hindlimb Unloading and Recovery Experiments

Cage ambulatory controls were followed for 14 d and were housed in groups of 2-3 mice/cage. For the disuse experiment, animals underwent hindlimb unloading via tail suspension (2 animals/cage) using a modified unloading method based on the traditional Morey-Holton design for studying disuse atrophy in rodents, with some additional modifications (37). Following day 14 of hindlimb unloading, animals were fasted for 5 hours and then euthanized for tissue analysis. In the Recovery from Disuse experiment, animals underwent 14 d of hindlimb unloading and afterwards were removed from the suspension apparatus then housed in individual cages for 7 days of ambulatory recovery. Body weight was monitored every other day for each experiment to ensure that mice were not experiencing excessive weight loss due to malnutrition or dehydration. Upon completion of each treatment group, the triceps surac muscle group (soleus, plantaris, gastrocnemius) from the left and right hindlimbs were carefully dissected, weighed, prepped for immunohistochemistry, and snap frozen in liquid nitrogen cooled isopentanc.

### STEM Characterization and Administration

STEM is a secretome biologic derived from partially differentiated embryonic stem cells composed of a multitude of factors involved in immunomodulation, cytoskeleton remodeling, and growth factor-mediated anabolic signaling. It is a sterile and nonpyrogenic aqueous solution of USP grade proteins, amino-acids, vitamins and minerals containing the factors secreted by partial differentiated pluripotent stem cells. The solution does not contain live cells, or any cell fragments, only the soluble secretions of cells. The factors were determined in three independent samples by a third party (RayBiotech) using a Quantibody Multiplex ELISA platform (Human Cytokine Array Q440). The measured quantities of the targets in each group were converted in picomoles (pmol/mL) and averaged for each group. FIG. 6 lists the measured factors that were highly expressed in the secretome. Factors were categorized using Reactome.

Pluripotent stem cells are expanded in GMP conditions using animal free reagents and recombinant growth factors (bFGF and Activin A) using ABSTEM (FUJIFILM Irvine Scientific). After expansion the growth factors are removed, after two days the cultures are exposed to a custom protein-free media containing 4.7% Trehaloze and USP grade salts, aminoacids, vitamins and buffers. The culture supernatant is collected daily and replaced with fresh media. The collected product over 7 days is pooled homogenized then filtered through a 0.22 um sterile membrane filter. The filtered product undergo a tangential flow filtration with a 1 kDa cutoff membrane for 100× volume reduction. The protein concentration is then standardized using a BCA assay and quantitative ELISA to 50 μg/mL Follistatin. Follistatin is a well-known antagonist of the myostatin and promotor of muscle growth. Sequences of follistatin protein and variants thereof are listed in FIG. 7. Follistatin and Follistatin variant sequences are listed in TABLE 1 below. Signal sequences have been excluded and Follistatin-N-terminal domains are underlined.

TABLE 1
Follistatin and variants (excluding signal peptide sequences; Follistatin-N-
terminal domain underlined).

follistatin isoform FST344 precursor [Homo sapiens] (NP_037541.1; SEQ ID NO: 1)
GNCWLRQAKNGRCQVLYKTELSKEECCSTGRLSTSWTEEDVNDNTLFKWMIENGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCIKAKSCEDIQCTGGKKCL
WDFKVGRGRCSLCDELCPDSKSDEPVCASDNATYASECAMKEAACSSGVLLEVKHSG
SCNSISEDTEEEEEDEDQDYSFPISSILEW
follistatin isoform FST317 precursor [Homo sapiens] (NP_006341.1; SEQ ID NO: 2)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCIKAKSCEDIQCTGGKKCL
WDFKVGRGRCSLCDELCPDSKSDEPVCASDNATYASECAMKEAACSSGVLLEVKHSG
SCN
follistatin isoform X1 [Homo sapiens] (XP_005248457.1; SEQ ID NO: 3)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCISRYSGLRKEKEKTEAKS
CEDIQCTGGKKCLWDFKVGRGRCSLCDELCPDSKSDEPVCASDNATYASECAMKEAA
CSSGVLLEVKHSGSCNSISEDTEEEEEDEDQDYSFPISSILEW
follistatin isoform X2 [Homo sapiens] (XP_011541401.1; SEQ ID NO: 4)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCISRYSGLRKEKEKTEAKS
CEDIQCTGGKKCLWDFKVGRGRCSLCDELCPDSKSDEPVCASDNATYASECAMKEAA
CSSGVLLEVKHSGSCNSISEDTEEEEEDEDQDYSFPISSILEW
follistatin isoform X3 [Homo sapiens] (XP_005248458.1; SEQ ID NO: 5)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCITKSCEDIQCTGGKKCLW
DFKVGRGRCSLCDELCPDSKSDEPVCASDNATYASECAMKEAACSSGVLLEVKHSGS
CNSISEDTEEEEEDEDQDYSFPISSILEW
follistatin isoform X4 [Homo sapiens] (XP_024310094.1; SEQ ID NO: 6)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCITKSCEDIQCTGGKKCLW
DFKVGRGRCSLCDELCPDSKSDEPVCASDNATYASECAMKEAACSSGVLLEVKHSGS
CNSISEDTEEEEEDEDQDYSFPISSILEW
follistatin isoform X5 [Homo sapiens] (XP_016864443.1; SEQ ID NO: 7)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCISRYSGLRKEKEKTEAKS
CEDIQCTGGKKCLWDFKVGRGRCSLCDELCPDSKSDEPVCASDNATYASECAMKEAA
CSSGVLLEVKHSGSCNSPLYSPI
follistatin isoform X6 [Homo sapiens] (XP_005248459.1; SEQ ID NO: 8)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCISRYSGLRKEKEKTEAKS
CEDIQCTGGKKCLWDFKVGRGRCSLCDELCPDSKSDEPVCASDNATYASECAMKEAA
CSSGVLLEVKHSGSCN
follistatin isoform X7 [Homo sapiens] (XP_005248460.1; SEQ ID NO: 9)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKTYRNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLAYEGKCITKSCEDIQCTGGKKCLW
DFKVGRGRCSLCDELCPDSKSDEPVCASDNATYASECAMKEAACSSGVLLEVKHSGS
CN
follistatin isoform X8 [Homo sapiens] (SEQ ID NO: XP_016864444.1; SEQ ID NO: 10)
MIFNGGAPNCIPCKETCENVDCGPGKKCRMNKKNKPRCVCAPDCSNITWKGPVCGLD
GKTYRNECALLKARCKEQPELEVQYQGRCKKTCRDVFCPGSSTCVVDQTNNAYCVTC
NRICPEPASSEQYLCGNDGVTYSSACHLRKATCLLGRSIGLAYEGKCISRYSGLRKE
KEKTEAKSCEDIQCTGGKKCLWDFKVGRGRCSLCDELCPDSKSDEPVCASDNATYAS
ECAMKEAACSSGVLLEVKHSGSCNSISEDTEEEEEDEDQDYSFPISSILEW
follistatin, isoform CRA_b [Homo sapiens] (EAW54881.1; SEQ ID NO: 11)
GNCWLRQAKNGRCQVLYKTELSKEECCSTGRLSTSWTEEDVNDNTLFKWMIFNGGAP
NCIPCKETCENVDCGPGKKCRMNKKNKPRCVCAPDCSNITWKGPVCGLDGKVYQ
FST [Homo sapiens] (CAG46612.1; SEQ ID NO: 12)
GNCWLRQAKNGRCQVLYKTELSKEECCSTGRLSTSWTEEDVNDNTLFKWMIFNGGAP
NCIPCKETCENVDCGPEKKCRMNKKNKPRCVCAPDCSNITWKGPVCGLDGKTYCNEC
ALLKARCKEQPELEVQYQGRCKKTCRDVFCPGSSTCVVDQTNNAYCVTCNRICPEPA
SSEQYLCGNDGVTYSSACHLRKATCLLGRSIGLVYEGKCIKAKSCEDIQCTGGKKCL
WDFKVGRGRCSLCDELCPDSKSDEPVCASDNATYASECAMKEAACSSGVLLEVKHSG
SCN
follistatin like 1 [ Homo sapiens] (NP_009016.1; SEQ ID NO: 13)
EEELRSKSKICANVFCGAGRECAVTEKGEPTCLCIEQCKPHKRPVCGSNGKTYLNHC
ELHRDACLTGSKIQVDYDGHCKEKKSVSPSASPVVCYQSNRDELRRRIIQWLEAEII
PDGWFSKGSNYSEILDKYFKNFDNGDSRLDSSEFLKFVEQNETAINITTYPDQENNK
LLRGLCVDALIELSDENADWKLSFQEFLKCLNPSFNPPEKKCALEDETYADGAETEV
DCNRCVCACGNWVCTAMTCDGKNQKGAQTQTEEEMTRYVQELQKHQETAEKTKRVST
KEI
Follistatin-N-terminal domains
TCENVDCGPGKKCRMNKKNKPRCV (SEQ ID NO: 14)
TCENVDCGPEKKCRMNKKNKPRCV (SEQ ID NO: 15)
CANVFCGAGRECAVTEKGEPTCL (SEQ ID NO: 16)

The analysis of the composition revealed in addition high amounts of Fetuin A, a multifunctional molecule that has a TGFb/BMP binding domain (https://escholarship.org/content/qt59r439q2/qt59r439q2_noSplash_a6f18bfc32c670e635ccc8c229 ba7cd2.pdf) inhibits TGFb1 and BMP2 activity and has mineral binding domains with a particular affinity for Ca, thus considered a Ca reservoir or as a calcification preventive structure. By inhibiting TGFb and BMP2, fetuin A can promote skeletal muscle differentiation (http://genesdev.cshlp.org/content/15/22/2950.abstract) (https://bmcdevbiol.biomedcentral.com/articles/10.1186/1471-213X-11-44). Fetuin A protein isoforms are listed in TABLE 2 below. Signal sequences have been excluded and TGF-beta binding domains are underlined.

TABLE 2
Fetuin A protein isoforms (excluding signal peptide sequences; TGF-beta binding
domain underlined).

Fetuin A isoform 1 (NP_001341500.1; SEQ ID NO: 17)
APHGPGLIYRQPNCDDPETEEAALVAIDYINQNLPWGYKHTLNQIDEVKVWPQQPSG
ELFEIEIDTLETTCHVLDPTPVARCSVRQLKEHAVEGDCDFQLLKLDGKFSVVYAKC
DSSPADSAEDVRKVCQDCPLLAPLNDTRVVHAAKAALAAFNAQNNGSNFQLEEISRA
QLVPLPPSTYVEFTVSGTDCVAKEATEAAKCNLLAEKQYGFCKATLSEKLGGAEVAV
TCMVFQTQPVSSQPQPEGANEAVPTPVVDPDAPPSPPLGAPGLPPAGSPPDSHVL
(Chain A, SEQ ID NO: 21)
LAAPPGHQLHRAHYDLRHTFMGVVSLGSPSGEVSHPRKTR (linker, SEQ ID NO: 22)
TVVQPSVGAAAGPVVPPCPGRIRHFKV (Chain B, SEQ ID NO: 23)
Fetuin A isoform 2 (NP_001613.2; SEQ ID NO: 18)
APHGPGLIYRQPNCDDPETEEAALVAIDYINQNLPWGYKHTLNQIDEVKVWPQQPSG
ELFEIEIDTLETTCHVLDPTPVARCSVRQLKEHAVEGDCDFQLLKLDGKFSVVYAKC
DSSPDSAEDVRKVCQDCPLLAPLNDTRVVHAAKAALAAFNAQNNGSNFQLEEISRAQ
LVPLPPSTYVEFTVSGTDCVAKEATEAAKCNLLAEKQYGFCKATLSEKLGGAEVAVT
CMVFQTQPVSSQPQPEGANEAVPTPVVDPDAPPSPPLGAPGLPPAGSPPDSHVL
(Chain A, SEQ ID NO: 24)
LAAPPGHQLHRAHYDLRHTFMGVVSLGSPSGEVSHPRKTR (linker, SEQ ID NO: 22)
TVVQPSVGAAAGPVVPPCPGRIRHFKV (Chain B, SEQ ID NO: 23)
Fetuin A isoform 3 (NP_001341501.1; SEQ ID NO: 19)
APHGPGLIYRQPNCDDPETEEAALVAIDYINQNLPWGYKHTLNQIDEVKVWPQPSGE
LFEIEIDTLETTCHVLDPTPVARCSVRQLKEHAVEGDCDFQLLKLDGKFSVVYAKCD
SSPDSAEDVRKVCQDCPLLAPLNDTRVVHAAKAALAAFNAQNNGSNFQLEEISRAQL
VPLPPSTYVEFTVSGTDCVAKEATEAAKCNLLAEKQYGFCKATLSEKLGGAEVAVTC
MVFQTQPVSSQPQPEGANEAVPTPVVDPDAPPSPPLGAPGLPPAGSPPDSHVL
(Chain A, SEQ ID NO: 25)
LAAPPGHQLHRAHYDLRHTFMGVVSLGSPSGEVSHPRKTR (linker, SEQ ID NO: 22)
TVVQPSVGAAAGPVVPPCPGRIRHFKV (Chain B, SEQ ID NO: 23)
Fetuin A isoform 4 (NP_001341502.1; SEQ ID NO: 20)
APHGPGLIYRQPNCDDPETEEAALVAIDYINQNLPWGYKHTLNQIDEVKVWPQQPSG
ELFEIEIDTLETTCHVLDPTPVARCSVRQLKEHAVEGDCDFQLLKLDGKFSVVYAKC
DSSPDSAEDVRKVCQDCPLLAPLNDTRVVHAAKAALAAFNAQNNGSNFQLEEISRAQ
LVPLPPSTYVEFTVSGTDCVAKEATEAAKCNLLAEKPVSSQPQPEGANEAVPTPVVD
PDAPPSPPLGAPGLPPAGSPPDSHVL (Chain A, SEQ ID NO: 26)
LAAPPGHQLHRAHYDLRHTFMGVVSLGSPSGEVSHPRKTR (linker, SEQ ID NO: 22)
TVVQPSVGAAAGPVVPPCPGRIRHFKV (Chain B, SEQ ID NO: 23)
TGF-beta binding domain (SEQ ID NO: 27)
CDFQLLKLDGKFSVVYAKC

Other myogenic agonists and antagonists found in the STEM composition are included in TABLE 3 and TABLE 4 below:

TABLE 3
Myogenic antagonist factors

| Myogenic | | | | Approxi- | |
|---|---|---|---|---|---|
| antagonist | SA100680 | SA100681 | SA100682 | mative | average |
| factors | (pg) | (pg) | (pg) | MW | pmol |
| IGFBP-3 | 107,265.8 | 118,634.1 | 97,839.3 | 31,674 | 3.407 |
| Dkk-3 | 39,884.9 | 35,960.0 | 39,189.3 | 36,300 | 1.056 |
| Periostin | 12,253.7 | 13,767.8 | 13,928.7 | 75,000 | 0.178 |
| TGFb1 | 3,820.8 | 6,042.3 | 6,515.4 | 25,000 | 0.218 |
| IL-6 | 1,511.2 | 2,105.0 | 2,690.5 | 21000 | 0.100 |
| FGF-21 | 340.4 | 483.6 | 618.1 | 19900 | 0.024 |
| TGFb2 | 317.4 | 525.2 | 984.5 | 25000 | 0.024 |
| TNFb | 71.0 | 126.3 | 110.7 | 18645 | 0.006 |
| TGFb3 | 1.2 | 20.2 | 15.9 | 47200 | 0.000 |
| Activin A | 0.0 | 208.9 | 0.0 | 26200 | 0.003 |
| LAP(TGFb1) | 856.2 | 953.1 | 751.2 | 30400 | 0.028 |
| TGFa | 99.2 | 119.8 | 62.3 | 11700 | 0.008 |

| | |
|---|---|
| Total | 5.052 |

TABLE 4
Pro-myogenic factors (agonists)

| Pro-myogenic | | | | Approxi- | |
|---|---|---|---|---|---|
| factors | SA100680 | SA100681 | SA100682 | mative | average |
| (agonists) | (pg) | (pg) | (pg) | MW | pmol |
| Fetuin A | 582,856.5 | 672,420.1 | 304,696.1 | 48,400 | 10.744 |
| OPN | 129,872.9 | 136,029.2 | 155,570.4 | 44,000 | 3.193 |
| IGFBP-4 | 125,591.3 | 133,417.6 | 109,953.8 | 26,800 | 4.589 |
| MMP-1 | 97,830.1 | 84,864.0 | 100,381.9 | 54,000 | 1.747 |
| TSP-1 | 86,652.9 | 72,765.4 | 80,479.5 | 450,000 | 0.178 |
| GROa / | 77,071.2 | 66,823.6 | 43,306.0 | 11,300 | 5.522 |
| CXCL1 | | | | | |
| Follistatin | 62,432.2 | 36,391.7 | 49,457.3 | 31,700 | 1.559 |
| MMP-10 | 60,700.1 | 43,130.1 | 44,950.7 | 43,000 | 1.153 |
| Follistatin- | 25,257.6 | 28,838.1 | 24,294.5 | 26,000 | 1.005 |
| like 1 | | | | | |
| bIG-H3 | 19,167.0 | 23,895.6 | 20,269.9 | 19,900 | 1.061 |
| hCGb | 16,033.8 | 74,766.7 | 12,884.2 | 22,500 | 1.536 |
| RGM-B | 11,779.9 | 10,268.8 | 9,075.3 | 40,000 | 0.259 |
| VEGF | 4,614.6 | 4,078.3 | 3,841.7 | 27000 | 0.155 |
| Cripto-1 | 4,479.1 | 4,495.9 | 4,332.0 | 28000 | 0.158 |
| HGF | 3,422.9 | 2,425.4 | 1,146.4 | 84000 | 0.028 |
| BMP-5 | 3,035.1 | 6,020.6 | 6,440.0 | 15600 | 0.331 |
| bFGF | 2,267.4 | 2,135.2 | 2,057.9 | 16500 | 0.131 |
| IGF-2 | 2,234.4 | 1,740.0 | 1,995.1 | 20140 | 0.099 |
| PDGF-AA | 2,055.4 | 2,122.1 | 2,101.9 | 28900 | 0.072 |
| FGF-19 | 1,417.0 | 1,691.4 | 1,195.8 | 21800 | 0.066 |
| WISP-1 | 954.9 | 1,897.6 | 1,354.1 | 45000 | 0.031 |

| | |
|---|---|
| Total | 33.617 |

The molar ratio of agonists and antagonists for muscle growth and differentiation averaged 6.65:1 on 3 different manufacturing lots. The dominance of the pro-myogenic factors conducted to theoretical indication for the use of the STEM for muscle regeneration.

Mice were given a 100 μl intramuscular injection of either PBS or STEM (2 μL STEM/100 μL PBS) into the right triceps surae muscle group every other day for each treatment group using a similar injection strategy as reported by Dumont and Frenette (38). Ambulatory control and hindlimb unloaded mice received a total of six injections over the respective 14-day period treatment group duration. To effectively examine STEM's effect on muscle recovery/regrowth, a total of 3 injections were given to the animals that underwent hindlimb unloading and recovery yet only delivered during the 7-day recovery phase of the treatment. All mice received the final treatment injection the day prior to being euthanized to avoid any acute treatment effects.

### Hindlimb Unloading and Recovery

For the hindlimb unloading and reload groups, animals underwent hindlimb unloading via tail suspension (2 animals/cage) using a modified unloading method based on the traditional Morey-Holton design for studying disuse atrophy in rodents, with some additional modifications (38). Body weight was monitored every other day to ensure that mice were not experiencing excessive weight loss due to malnutrition or dehydration. Following day 14 of hindlimb unloading, animals were fasted for 5 hours and then euthanized for tissue analysis. In a separate group of mice, animals underwent 14 d of hindlimb unloading and afterwards were removed from the suspension apparatus then housed in individual cages for 7 days of ambulatory recovery. Cage ambulatory controls were followed for 14 d and were housed in groups of 2-3 mice/cage. Upon completion of each treatment group, the triceps surae muscle group (soleus, plantaris, gastrocnemius) from the left and right hindlimbs were carefully dissected, weighed, prepped for immunohistochemistry, and snap frozen in liquid nitrogen cooled isopentane.

### Grip Strength

To assess whole body strength, mice underwent grip strength analysis on a rodent grip strength meter (Columbus Instruments, Columbus OH). Mice grasped the force transducer grid with their forelimbs and hindlimbs and were gently pulled by the tail across the grid by the same investigator. Five repetitions with a five second rest period were averaged to determine each animal's grip strength. In the ambulatory control mice, grip strength was determined twice: the day prior to initiating PBS or STEM treatment and on day 13 one day prior to tissue harvest. The Disuse experimental mice were tested on three occasions: one day prior to treatment, at day 7 and again on day 13 one day prior to tissue harvest. The Recovery from Disuse experimental mice were also tested on three occasions: one day prior to hindlimb unloading, at the end of hindlimb unloading, and on day 6 of the recovery period (FIG. 1). All measurements were taken prior to the injection to avoid confounding effects of the injection.

### Immunofluorescence

The right (treated) soleus muscle was prepped for histology by embedding in OCT and freezing in liquid nitrogen cooled isopentane. Several cryosections were cut at a thickness of 10 μm and used for staining myofiber cross sectional area (CSA) using cell membrane (laminin or dystrophin (1:100; Santa Cruz Biotechnology, Dallas, TX)). Myofiber CSA was measured using semiautomatic muscle analysis with segmentation of histology, a MATLAB application (SMASH) alongside ImageJ software (25, 39). To assess macrophage abundance, primary antibodies anti-rat CD68 (pro-inflammatory-like, 1:100 Bio-Rad, Hercules, CA), and anti-rabbit CD163 (anti-inflammatory-like, 1:100, Bio-Rad, Hercules, CA) were used. Anti-rat secondary antibody (1:250, AF555, Invitrogen), anti-mouse secondary antibody (1:500, A488, Invitrogen) and anti-rabbit secondary antibody (1:500. AF647, Invitrogen) were applied and then mounted in DAPI-containing mounting medium (Vector). Muscle satellite cells were stained using Pax7 (1:1000, Cell Signaling, Danvers, MA). To assess fibrosis, cryosections were stained using biotin collagen hybridizing peptide (1:100, BCHP-marker for collagen breakdown, 3Helix, Salt Lake City, UT) and Collagen IV (1:100, COLIV-marker for collagen synthesis, Abcam, Cambridge, MA). The ratio of these two stains is an indicator of overall collagen turnover and thus fibrosis of the muscle. All stained slides were observed with a fully automated wide-field light microscope (Nikon, Tokyo, Japan) with the ×10 or ×20 objective lens. Images were taken using a high sensitivity Clara CCD camera (Belfast, UK).

### C2C12 Myogenic Index, Myofiber Size, and RNA Sequencing

C2C12 myoblasts were plated in 6 well plates and upon achieving ˜ 95% confluency, differentiation media DMEM supplemented with 2% horse serum and penicillin/streptomycin was used for 5 days to differentiate myoblasts to myotubes. Upon completion of 5 days, differentiation media was removed and replaced with media that contained varying percentages of STEM (2, 4, 8, 12, and 24%) instead of horse serum for 24 h. Control wells contained either normal differentiation media containing 2% horse serum or 4% horse serum. A serum free control was also utilized. Following 24 h, cells were fixed and stained using MF 20 (DSHB, Iowa City, Iowa) and DAPI. Cells were then imaged at the University of Utah Imaging Core facility on a Nikon Eclipse Ti widefield scanning microscope. A 10×10 field image was obtained for each well and analyzed using ImageJ (NIH, Bethesda, MD). Myonuclear fusion is defined as nuclei in myotube/total nuclei in image field. Myotube percent area was defined as percent area of image covered by myotubes.

A separate group of cells utilizing 4% horse serum as control and 4% STEM were collected for total RNA using 1 ml of Qiazol per well and purified (using miRNAcasy Mini Kit). RNA was then treated with TURBO DNase (ThermoFisher) and purified using RNA Clean and Concentrator 5 Columns (Zymo Research). Libraries were prepared with Illumina TruSeq Stranded Total RNA Library Prep Ribo-Zero Gold (Genome Builds mm10, M_musculus_Dec_2011, GRCm38) and RNA was sequenced using Illumina NovaSeq Reagent Kit v1.5 150×150 bp Sequencing (100 M read-pairs).

### Statistical Analysis and Bioinformatic Analysis.

Results are reported as the means±standard error. A T-Test, One-Way ANOVA, or Two-Way ANOVA with repeated measures was employed when appropriate. Post-hoc analyses were performed with Tukey or Student-Newman-Keuls methods when appropriate. The accepted level of significance was set at p<0.05 for all analysis. Statistical analysis was performed using Prism GraphPad 7 (GraphPad Software Inc., La Jolla, CA). For RNA sequencing, differentially expressed genes were identified using a 5% false discovery rate with DESeq2 version 1.26.00. Data can be found on the Gene Expression Omnibus. The volcano plot was generated by taking the −Log 10 (Adj. P-value) on the Y axis and plotting vs the Log 2 fold change on the X axis. The top 20 significantly decreased and top 20 increased genes were identified by taking all significantly altered transcripts (Adj. P-Value ≤0.05) and then sorting by log 2 fold change. Values were converted out of log 2 for presentation in the table.

### Results

### Effect of STEM on Soleus Muscle and Strength in Ambulatory Control Mice

One goal was to determine the effect of STEM on hindlimb muscle mass and strength during 14 days of normal cage ambulation in old mice. The soleus was examined due to its sensitivity to muscle atrophy during disuse and, secondly, during the course of our experiments it was the muscle most influenced by STEM (e.g., plantaris size was increased with STEM while gastrocnemius was not affected). Strikingly, STEM delivery to a single limb over the course of 14 days of normal cage ambulation increased whole body grip strength (p=0.008) above baseline levels (FIG. 2A). Moreover, STEM increased soleus muscle mass (p=0.0001) above PBS treated mice (FIG. 2B). Consistent with an increase in soleus muscle mass, STEM also increased (p=0.02) fiber cross-sectional area (CSA) (FIG. 2C). Moreover, STEM increased BCHP and decreased COLIV (data not shown) resulting in an increase in BCHP/COLIV ratio (p=004) (FIG. 2D) suggesting a decrease in overall soleus muscle collagen content.

Given the STEM cocktails unique immunomodulatory composition, it was next determined if the treatment influenced muscle pro- and anti-inflammatory-like macrophages in the soleus muscle. Immuno-staining of cryosections showed that STEM increased (p=0.0002) CD68+/DAPI+ cells (pro-inflammatory M1-like macrophages) and (p=0.0002) CD163+/DAPI+ (anti-inflammatory M2-like macrophages) macrophage sub-types (FIG. 2E). Furthermore, STEM tripled Pax7+ cell content (p=0.0001) in ambulatory control mice compared to PBS (FIG. 2F). Together, these results suggest that STEM improved soleus muscle size, strength, reduced muscle fibrosis, increased macrophage infiltration, and increased Pax7+ cells during 14 days of cage ambulation in aged mice.

### Effect of STEM on Soleus Muscle and Strength During Hindlimb Unloading

The second experiment determined if STEM administration would mitigate muscle atrophy and weakness, which is modeled as a result of 14 days of hindlimb unloading. Following 14 days of hindlimb unloading, STEM treatment was found to completely prevent the loss of grip strength at 7- and 14-days HU when compared to HU mice treated with PBS (p=0.004) (FIG. 3A). Similarly, STEM treatment prevented soleus muscle atrophy during HU (p=0.006) (FIG. 3B). In agreement with the muscle mass data, STEM attenuated fiber CSA atrophy (p=0.002) in the soleus muscle compared to PBS (FIG. 3C). STEM also increased BCHP and decreased COLIV (data not shown), which lead to an increased ratio of BCHP to COLIV (p=0.04) in soleus muscle of HU mice (FIG. 3D). Finally, STEM injection of experimental mice did not affect body weight compared to PBS treatment (data not shown). These results demonstrate that STEM is capable of mitigating disuse induced atrophy and improving collagen turnover in aged soleus muscle.

It was next determined if STEM modulated macrophage infiltration and Pax7 cell content in unloaded muscle. STEM was found to increase (p=0.0009) CD68+/DAPI+ cells (pro-inflammatory M1-like macrophages) but not CD163+/DAPI+ cells (anti-inflammatory M2-like macrophages) in the soleus muscle compared to PBS-treated HU mice (FIG. 3E). Lastly, it was determined that STEM doubled the Pax7+ cell content in soleus muscle (p=0.0004) when compared to PBS control during HU (FIG. 3F). Together, these results suggest that STEM is capable of increasing pro-inflammatory macrophages in aged soleus muscle during disuse atrophy while also increasing the total satellite cell (Pax7+) content.

Effect of STEM on Recovery of Soleus Muscle and Strength after Disuse

It was next determined if STEM would enhance the recovery of muscle and strength following disuse atrophy (14-days of HU). It was found that as little as three intramuscular injections of STEM over the course of 7 days of recovery, grip strength was completely restored to baseline levels (p=0.0001) and was in contrast to the PBS treatment group (FIG. 4A). Moreover, STEM restored soleus muscle mass (p=0.001) compared to PBS treated mice (FIG. 4B). Similar to the muscle mass data, STEM increased soleus fiber CSA (p=0.003) above the PBS treated mice (FIG. 4C) and was similar to the fiber CSA of ambulatory control mice from the first experiment. STEM also reduced the accumulation of collagen noted by an increased ratio (p=0.0009) of BCHP/COLIV (FIG. 4D) which was again due to an increase in BCHP and a decrease in COLIV (data not shown). These results demonstrate that STEM improved aged soleus muscle mass and promoted the growth of myofibers during recovery from disuse atrophy. Additionally, STEM appeared to increase collagen turnover and an overall net decrease in collagen content.

Finally, the effect of STEM on muscle macrophages and satellite cells during recovery from disuse was examined. STEM increased (p=0.0056) CD68+/DAPI+ cells (pro-inflammatory M1-like macrophages) in the soleus muscle but not CD163+/DAPI+ cells (anti-inflammatory M2-like macrophages) during 7 days of recovery when compared to PBS treated mice (FIG. 4E). STEM also doubled the Pax7+ cells (p=0.0005) in the soleus muscle of mice during 7 days of recovery when compared to PBS treated mice (FIG. 4F). Together, these results demonstrate that STEM increased macrophage infiltration and enhanced the satellite cell pool in aged muscle during recovery.

### Effect of STEM on C2C12 Myotube Fusion and Size

To further evaluate the effect of STEM independently on skeletal muscle, C2C12 myotubes were subjected to varying concentrations of STEM after 5 days of differentiation. A robust induction (p=0.0001) of myonuclear fusion (percentage of nuclei per plate in a myotube) and myotube percent area (size; p=0.0002) at 2, 4, 8, and 12% STEM was noted when compared to controls (2 and 4% horse serum as well as serum free media) (FIG. 5A). In lieu of these results, the experiments were repeated in a separate cohort of C2C12 myotubes using 4% horse serum as our control and 4% STEM as our treatment group and performed RNA sequencing analysis in order to provide insight into how STEM may affect C2C12 at the molecular level. STEM was found to induce a transcriptional program related to cell growth and remodeling of the extracellular matrix (FIG. 5B). Notable genes that were regulated pertained to collagen synthesis, degradation, and growth factors (IGF binding protein 3, Col23a1, Col14a1, Mt2, Mt1, and Fmod). Overall, these results suggest that STEM improved myogenesis in C2C12 myotubes through the regulation of genes related to growth factors and promotion of extracellular matrix remodeling.

### DISCUSSION

STEM was found to attenuate the loss of soleus muscle mass and fiber cross-sectional area during disuse while also increasing mass and cross-sectional after only 7 days of recovery in aged mice. Moreover, these findings were supported by improved grip strength and muscle collagen turnover. STEM's unique secretome composition was also found to be capable of immunomodulation as noted by increased infiltration of muscle macrophages and an accumulation of satellite cells across all treatment groups. Finally, in muscle cell culture experiments, STEM was shown to have a potent effect on myotube size and fusion while also promoting a transcriptional signature noted by robust extracellular matrix remodeling. Together, these results suggest that STEM is a novel therapeutic that can enhance muscle size and strength and reduce fibrosis during disuse and recovery in aged mice.

Skeletal muscle is a highly adaptive tissue capable of modulating its size and functional capacity in response to changes in loading stimuli (40). Muscle disuse in rodents induced by hindlimb unloading induces robust atrophy and weakness and is frequently used to model disuse atrophy and recovery in humans (1, 3, 10, 41). While aging does not accelerate muscle mass loss during disuse (42, 43), aging worsens the recovery following such an insult (43-45) and is especially concerning for aged individuals who have low muscle reserve such as those with frailty and sarcopenia. These impairments can have serious ramifications since limited muscle size and functional reserve can lead to decreased quality of life (45). The primary findings discussed herein demonstrate that STEM administration was capable of increasing soleus muscle mass and cross-sectional area in a variety of age-related conditions: increasing soleus size after 2 weeks in ambulatory control aged mice, blunting disuse atrophy and amplifying muscle size during recovery all of which have application to sarcopenia, muscle disuse, and age-related impairments following recovery from disuse. Interestingly, whole body grip strength was improved across all treatment groups. This is in spite that STEM was given intramuscularly to a single limb, suggesting that a systemic effect may have occurred through diffusion into circulation. Furthermore, in-vitro analysis demonstrated that STEM can influence muscle directly by inducing myotube hypertrophy and fusion when compared to horse serum controls. Given STEM's varying composition of growth factors (see tables in the methods) such as follistatin, insulin, IGF-Binding Protein 2, and IGF-Binding Protein-6 (46-50), it is logical that this compound was capable of promoting hypertrophy.

Another major finding was that STEM increased muscle collagen turnover (thus reduced fibrosis) and increased a pro-inflammatory-like macrophage infiltration in all three experiments. A common ailment of aged skeletal muscle is elevated collagen content which is widely believed to contribute to impaired function and regrowth from disuse atrophy (11, 12, 51, 52). Recent evidence suggests that an aged immune system may contribute to the impairment of skeletal muscle both during disuse and recovery (22, 23, 25, 52). Dysfunction in the resident pro-inflammatory and an accumulation of anti-inflammatory macrophages may underscore the poor remodeling of aged skeletal muscle (22, 23, 25). Pro-inflammatory-like macrophages secrete a specific cytokine profile which increase satellite cell proliferation and inhibit myoblast differentiation (53-55). On the other hand, anti-inflammatory macrophages secrete cytokine and growth factors that promote myoblast differentiation and inhibit proliferation 54, 56) while also increasing collagen synthesis (57). Moreover, anti-inflammatory macrophages encourage satellite cells to shift toward a more pro-fibrotic phenotype in aged muscle (19, 21, 22). In the study, it was demonstrated that STEM increased Cd68+ pro-inflammatory-like macrophages across all experimental treatment groups. This is in line with the success of immunotherapies that enhance pro-inflammatory macrophage infiltration in healthy and aged skeletal muscle and thus are effective to enhance muscle regrowth from disuse and injury (58-60). These findings suggest that STEM administration during disuse and recovery promoted infiltration of pro-inflammatory macrophages and may be related to the increased collagen turnover and an overall less fibrotic muscle. The transcriptome analysis in C2C12 myotubes treated with STEM also support profound changes in gene networks that are related to skeletal muscle remodeling and collagen synthesis. The infiltration of macrophages with the administration of STEM is a testament to the various cytokines and immune cell modulating molecules present in the cocktail. While the only immune cells examined were macrophages, the cocktail may be indirectly influencing muscle by regulating a multitude of immune cells important for muscle regrowth (e.g., neutrophils, T-cells).

Finally, STEM administration was found to robustly increase the total amount of Pax7+ cells in aged muscle across all treatment groups. Satellite cells are critical for skeletal muscle maintenance and regeneration following damage and are reduced or dysfunctional in aging skeletal muscle (61-63). A recent study highlighted the aging immune system as a modulator of the muscle stem cell environment (22). For example, transplantation of old bone marrow cells into young mice decreased skeletal muscle Pax 7+ cells and biased them toward a more fibrogenic cellular lineage (22). Therefore, these results when combined with the macrophage observations, suggest that STEM may regulate the aged skeletal muscle immune cells thereby improving the muscle microenvironment and the satellite cell niche.

In summary, the results discussed herein suggest that the secretome, STEM, improved aged skeletal muscle mass and strength and during disuse atrophy and recovery. While an exact mechanism of action is difficult to determine, STEM's unique anabolic and pro-myogenic growth factor properties and cytokine profile may modulate immune and satellite cells that could possibly influence collagen turnover. STEM was also found to have isolated benefits in soleus compared to the other hindlimb muscle groups. Though STEM may preferentially target soleus muscle due to the muscles' sensitivity to insulin and growth factors (64-66) and high macrophage content (67), some improvements in plantaris size (but not gastrocnemius) in ambulatory control mice and during disuse atrophy with STEM treatment were noted. This suggests that further optimization of dosing and delivery approaches will be necessary to advance STEM as a future therapeutic in aging muscle.

### Quantitative Assessment of STEM Components

Samples of 3 lots of STEM produced by standardized manufacturing methods were sent to a third party research organization for multiplex ELISA assay. The assay used Quantikine arrays and the work was performed under GLP by RayBiotech. A total of 400 targets were analyzed including growth factors, cytokines, chemokines, enzymes, and extracellular matrix components. The results of multiplex ELISA performed by RayBiotech are included in TABLE 5.

TABLE 5
Exploratory evaluation of the STEM composition.
Only targets that produced measurable amounts
are included (>1 pg/mL). Values are in pg/mL

| Code | SA100680 | SA100681 | SA100682 |
|---|---|---|---|
| Activin A | 184.51 | 0.00 | 38.09 |
| ADAM12 | 0.00 | 402.67 | 0.00 |
| ADAM8 | 6525.09 | 7369.50 | 222.12 |
| Adipsin | 0.00 | 106.73 | 0.00 |
| aFGF | 1127.96 | 18.18 | 939.25 |
| AFP | 4142.53 | 4248.52 | 1.98 |
| Aggrecan | 0.00 | 6.64 | 0.00 |
| Albumin | 8778.77 | 4280.99 | 7482.56 |
| ALCAM | 75.13 | 66.61 | 85.54 |
| ANG-1 | 14.14 | 8.74 | 24.81 |
| ANG-2 | 0.00 | 32.86 | 41.87 |
| ANG-4 | 280.76 | 156.54 | 0.00 |
| Angiogenin | 153.29 | 369.02 | 31.31 |
| Angiostatin | 437.35 | 367.60 | 22.48 |
| ANGPTL4 | 0.00 | 262.38 | 0.00 |
| AR | 15.52 | 19.17 | 18.97 |
| Axl | 0.13 | 60.83 | 1.54 |
| B2M | 936.30 | 725.96 | 726.71 |
| B7-H3 | 188.14 | 98.51 | 147.13 |
| BCAM | 46.03 | 1492.54 | 101.58 |
| bFGF | 213.15 | 79.56 | 766.87 |
| bIG-H3 | 3139.76 | 5116.20 | 137.27 |
| BMP-4 | 36.65 | 0.00 | 40.75 |
| BMP-5 | 0.00 | 329.67 | 92.97 |
| BMP-7 | 0.00 | 0.00 | 27.91 |
| BMPR-IA | 0.00 | 1343.55 | 0.00 |
| BMPR-IB | 1679.21 | 1212.13 | 0.00 |
| b-NGF | 0.00 | 3.40 | 0.00 |
| CA125 | 234.91 | 302.14 | 297.59 |
| CA19-9 | 378.36 | 63.08 | 0.00 |
| CA9 | 48.16 | 0.00 | 0.00 |
| Cadherin-13 | 224.52 | 1113.50 | 0.00 |
| Cadherin-4 | 0.00 | 5555.06 | 0.00 |
| Cathepsin B | 254.49 | 738.10 | 127.66 |
| Cathepsin L | 132.17 | 931.55 | 1079.82 |
| Cathepsin S | 2.30 | 0.00 | 0.00 |
| CD155 | 17536.39 | 62525.27 | 0.00 |
| CD229 | 0.00 | 79.33 | 0.00 |
| CD23 | 0.00 | 0.00 | 46.21 |
| CD48 | 59935.82 | 182617.54 | 16325.84 |
| CD58 | 19288.40 | 112957.35 | 0.00 |
| CD99 | 64.75 | 71.33 | 18.09 |
| CEACAM-5 | 219.23 | 489.25 | 0.00 |
| CF XIV | 448.13 | 1335.43 | 575.31 |
| Chemerin | 0.00 | 6313.34 | 0.00 |
| CHI3L1 | 130.22 | 519.84 | 0.00 |
| Clusterin | 197.56 | 257.46 | 95.29 |
| Contactin-2 | 4310.09 | 111.43 | 209.38 |
| Cripto-1 | 214.26 | 0.00 | 115.09 |
| CXCL16 | 115.31 | 530.66 | 87.28 |
| Cystatin B | 1502.51 | 1043.38 | 1654.41 |
| Cystatin C | 12.86 | 99.96 | 0.00 |
| Cystatin EM | 569.41 | 2304.39 | 0.00 |
| Decorin | 12944.25 | 649.74 | 0.00 |
| Desmoglein 2 | 138.18 | 6372.29 | 0.00 |
| DKK-1 | 0.00 | 214.81 | 0.00 |
| Dkk-3 | 1734.43 | 2523.35 | 0.00 |
| Dkk-4 | 1.84 | 0.00 | 477.82 |
| DLL1 | 0.00 | 39.64 | 0.00 |
| DPPIV | 738.14 | 29159.48 | 0.00 |
| DR3 | 261.34 | 4043.26 | 0.00 |
| Dtk | 0.00 | 1214.96 | 89.03 |
| E-Cadherin | 15.51 | 52.21 | 7.52 |
| EGF | 0.00 | 0.20 | 0.51 |
| EGF R | 35.07 | 54.67 | 81.22 |
| EG-VEGF | 7.08 | 5.47 | 11.26 |
| EMMPRIN | 1331.58 | 1489.13 | 2559.33 |
| ENA-78 | 10.87 | 0.00 | 756.78 |
| EpCAM | 28.21 | 221.54 | 95.07 |
| Epo R | 0.00 | 0.00 | 189.55 |
| ErbB3 | 167.44 | 2243.72 | 931.73 |
| ErbB4 | 21.23 | 57.04 | 0.00 |
| ESAM | 0.00 | 2199.60 | 0.00 |
| FAP | 14.64 | 0.00 | 0.00 |
| FAS L | 2.78 | 0.00 | 0.00 |
| Fcg RIIBC | 148.31 | 0.00 | 72.09 |
| Ferritin | 67172.98 | 67341.90 | 69572.93 |
| Fetuin A | 115918.53 | 152409.95 | 547195.08 |
| FGF-21 | 0.00 | 49.65 | 0.00 |
| FGF-4 | 0.00 | 0.00 | 35.79 |
| Follistatin | 558.24 | 4132.84 | 2645.20 |
| FOLR1 | 1387.54 | 0.00 | 0.00 |
| Furin | 30.27 | 0.00 | 163.19 |
| Galectin-1 | 565.06 | 173.38 | 452.24 |
| Galectin-2 | 870.99 | 1934.70 | 0.00 |
| Galectin-7 | 2.45 | 1.62 | 0.88 |
| Galectin-9 | 4.26 | 54.77 | 22.15 |
| Gas 1 | 331.90 | 0.00 | 0.00 |
| GCP-2 | 569.89 | 0.00 | 205.88 |
| G-CSF R | 1007.24 | 0.00 | 0.00 |
| GDF-15 | 463.63 | 1710.82 | 544.82 |
| GH | 2.76 | 70.36 | 42.44 |
| gp130 | 157.07 | 149.20 | 8.24 |
| GRO | 126.72 | 180.48 | 82.21 |
| GROa | 6000.84 | 11123.51 | 3093.36 |
| HAI-2 | 134.65 | 0.00 | 1140.39 |
| hCGb | 0.00 | 21614.16 | 210.67 |
| HGF | 53.27 | 30.47 | 9.29 |
| HGF R | 0.00 | 551.41 | 92.62 |
| I-309 | 0.00 | 0.00 | 3.63 |
| ICAM-1 | 458.63 | 228.27 | 797.00 |
| ICAM-2 | 1.74 | 0.00 | 30.74 |
| ICAM-3 | 3.41 | 0.00 | 0.00 |
| ICOS | 880.70 | 3681.72 | 0.00 |
| IFNg | 0.00 | 0.00 | 5.11 |
| IGFBP-1 | 8.96 | 0.00 | 26.48 |
| IGFBP-2 | 2736.13 | 4522.66 | 2675.32 |
| IGFBP-3 | 1838.48 | 22985.79 | 0.00 |
| IGFBP-4 | 0.00 | 0.00 | 3013.62 |
| IGFBP-6 | 2157.04 | 2570.58 | 975.93 |
| IL-1 F6 | 0.00 | 0.00 | 816.53 |
| IL-1 F9 | 0.00 | 0.00 | 203.70 |
| IL-1 R3 | 2.51 | 14.36 | 0.09 |
| IL-11 | 0.00 | 27.72 | 24.33 |
| IL-13 R1 | 60.63 | 86.81 | 3.63 |
| IL-13 R2 | 16.76 | 0.00 | 0.00 |
| IL-15 | 0.00 | 2.45 | 4.24 |
| IL-15 R | 27.09 | 0.00 | 0.00 |
| IL-16 | 0.00 | 0.00 | 12.66 |
| IL-17B | 2.24 | 0.00 | 3.04 |
| IL-17E | 0.00 | 0.00 | 7.34 |
| IL-1a | 0.00 | 14.66 | 23.28 |
| IL-1ra | 0.00 | 53.14 | 4.74 |
| IL-2 | 0.00 | 1.67 | 3.90 |
| IL-2 Ra | 9.62 | 8.06 | 0.00 |
| IL-21 | 0.00 | 243.22 | 0.00 |
| IL-27 | 0.00 | 0.00 | 41.82 |
| IL-31 | 0.00 | 0.00 | 4.19 |
| IL-34 | 74.64 | 0.00 | 0.00 |
| IL-5 Ra | 203.83 | 0.00 | 0.00 |
| IL-6 | 1.78 | 58.86 | 11.96 |
| IL-6R | 6.52 | 13.22 | 20.49 |
| IL-8 | 9.09 | 1.14 | 6.40 |
| IL-9 | 0.00 | 0.00 | 1199.06 |
| Insulin | 241.60 | 278.57 | 519.17 |
| IP-10 | 27.79 | 0.00 | 0.00 |
| I-TAC | 6.23 | 0.00 | 1.33 |
| JAM-A | 10.71 | 148.42 | 0.00 |
| JAM-B | 640.25 | 1745.46 | 0.00 |
| Kallikrein 5 | 33.33 | 52.74 | 22.60 |
| LAP(TGFb1) | 31.17 | 10.41 | 343.61 |
| LDL R | 17.06 | 14.05 | 166.67 |
| Legumain | 271.30 | 106.52 | 2257.96 |
| Leptin R | 13.99 | 0.00 | 0.00 |
| LIF | 0.00 | 0.00 | 12.74 |
| Lipocalin-2 | 19.96 | 5.57 | 33.05 |
| LOX-1 | 37.44 | 0.00 | 33.30 |
| LRP-6 | 0.00 | 390.00 | 0.00 |
| LYVE-1 | 0.06 | 0.00 | 10.00 |
| MCP-1 | 274.17 | 327.34 | 64.33 |
| MCP-2 | 1.57 | 0.00 | 0.00 |
| MCP-4 | 1.01 | 0.75 | 0.47 |
| MCSF | 2.23 | 34.37 | 0.00 |
| MCSF R | 0.00 | 0.00 | 7.86 |
| MDC | 1.58 | 0.00 | 6.17 |
| MEPE | 1.66 | 0.00 | 0.00 |
| MICA | 0.00 | 0.00 | 4.23 |
| Midkine | 4413.14 | 6994.04 | 3377.79 |
| MIF | 11556.19 | 19376.89 | 23372.66 |
| MIP-1b | 0.00 | 0.78 | 0.34 |
| MIP-1d | 1.08 | 0.00 | 0.00 |
| MIP-3a | 1.78 | 0.25 | 0.16 |
| MMP-1 | 4931.78 | 629.12 | 0.00 |
| MMP-10 | 93.19 | 163.04 | 0.32 |
| MMP-13 | 69.17 | 56.03 | 45.04 |
| MMP-2 | 0.00 | 0.00 | 1105.63 |
| MMP-7 | 0.00 | 0.00 | 231.38 |
| MMP-9 | 1038.18 | 37982.63 | 2611.97 |
| NAP-2 | 16.12 | 0.00 | 59.24 |
| NCAM-1 | 534.97 | 0.00 | 0.00 |
| NGF R | 0.00 | 10.39 | 8.73 |
| Nidogen-1 | 9845.49 | 11603.50 | 812.37 |
| NOV | 665.43 | 0.00 | 16.30 |
| NrCAM | 21.56 | 0.00 | 0.00 |
| NSE | 14094.27 | 9168.99 | 11555.32 |
| NT-3 | 9.83 | 0.00 | 6.87 |
| NT-4 | 23.40 | 0.00 | 46.50 |
| OPG | 35.10 | 12.96 | 15.33 |
| OPN | 6386.22 | 8234.30 | 9860.98 |
| PAI-1 | 17538.26 | 18586.70 | 68790.73 |
| P-Cadherin | 1266.39 | 22156.56 | 9088.12 |
| PDGF-AA | 5.05 | 115.64 | 80.78 |
| PDGF-AB | 0.00 | 0.00 | 2.17 |
| PDGF-BB | 1.75 | 59.63 | 100.45 |
| Pentraxin 3 | 3140.94 | 524.54 | 41.29 |
| Pepsinogen I | 341.99 | 0.00 | 0.00 |
| Periostin | 3055.10 | 5040.79 | 17198.65 |
| PF4 | 48.88 | 0.00 | 12.54 |
| PIGF | 4.17 | 118.54 | 2.99 |
| Pref-1 | 1484.47 | 8061.94 | 1671.14 |
| PSMA | 222.41 | 0.00 | 440.45 |
| RAGE | 0.00 | 11.70 | 14.49 |
| RBP4 | 283.38 | 0.00 | 0.00 |
| Renin | 0.04 | 10.35 | 0.00 |
| Resistin | 407.50 | 0.00 | 0.00 |
| RGM-B | 33.80 | 3.83 | 0.00 |
| SCF | 0.00 | 6.52 | 0.00 |
| SCF R | 10.74 | 116.39 | 79.78 |
| Serpin A4 | 34.88 | 143.86 | 414.22 |
| SFRP-3 | 4985.83 | 6484.50 | 0.00 |
| Shh-N | 0.00 | 166.98 | 0.00 |
| Siglec-10 | 579.48 | 1150.46 | 0.00 |
| SLAM | 5.26 | 4198.06 | 0.00 |
| SP-D | 188.50 | 166.87 | 0.00 |
| Syndecan-1 | 106.59 | 46.74 | 1390.03 |
| Syndecan-4 | 440.76 | 1392.73 | 539.58 |
| TARC | 0.00 | 2.36 | 0.00 |
| Testican 2 | 1247.68 | 23519.15 | 0.00 |
| TF | 31.10 | 2.82 | 64.67 |
| TFPI | 1666.87 | 6476.97 | 6803.33 |
| TGFa | 0.44 | 2.95 | 2.58 |
| TGFb1 | 76.62 | 181.97 | 81.18 |
| TGFb2 | 153.53 | 0.00 | 0.00 |
| Thrombomodulin | 490.12 | 0.00 | 0.00 |
| Thrombospondin-2 | 341.41 | 0.00 | 85.06 |
| Tie-2 | 3.29 | 13.89 | 15.69 |
| TIM-1 | 0.00 | 51.59 | 0.00 |
| TIM-3 | 0.00 | 190.56 | 0.00 |
| TIMP-1 | 1542.16 | 1791.43 | 895.72 |
| TIMP-2 | 2540.67 | 4391.79 | 327.63 |
| TLR4 | 54.80 | 325.13 | 0.00 |
| TNF RI | 0.00 | 1132.08 | 280.54 |
| TNF RII | 0.00 | 195.97 | 9.05 |
| TNFa | 0.00 | 1.64 | 5.18 |
| TNFb | 0.71 | 4.71 | 6.64 |
| TRAIL | 0.09 | 0.19 | 0.00 |
| TRAIL R2 | 23.10 | 3.90 | 0.00 |
| TRAIL R3 | 0.00 | 62.43 | 0.00 |
| Transferrin | 84.99 | 0.00 | 0.00 |
| Trappin-2 | 0.00 | 0.00 | 6.85 |
| TSLP | 1.29 | 0.00 | 0.00 |
| TSP-1 | 1610.13 | 4797.57 | 0.00 |
| TWEAK | 36.44 | 0.00 | 66.79 |
| ULBP-1 | 100.23 | 117.46 | 0.00 |
| uPA | 47.99 | 0.51 | 0.00 |
| uPAR | 0.00 | 197.23 | 0.00 |
| VEGF | 2.35 | 40.13 | 2.92 |
| VEGF R1 | 3655.74 | 3524.27 | 7610.74 |
| VEGF R2 | 6.59 | 0.00 | 0.00 |
| VEGF R3 | 23.82 | 40.50 | 16.41 |
| VEGF-D | 0.00 | 2.29 | 0.00 |
| WISP-1 | 6745.95 | 7.75 | 2376.12 |

Samples from three additional lots that underwent concentration by tangential flow filtration (TFF) were further tested using specific ELISA kits for Follistatin, Fetuin A, and Activin A. The ELISA test was performed using two replicates for the standards and 3 replicates for each sample. The results are presented in TABLE 6.

TABLE 6
Quantitative ELISA of TFF concentrated samples.

| | | Follistatin | Activin A |
|---|---|---|---|
| Lot # | Fetuin A (ng/mL) | (ng/mL) | (pg/mL) |
| 20002 | 223.86 | 17.65 | Not detectable |
| 20003 | 213.50 | 38.25 | Not detectable |
| 20004 | 200.32 | 16.79 | Not detectable |

### Potency Assay

The pro-myogenic effect of the STEM could be theoretically attributed to its content in pro-myogenic factors such as Follistatin. These factors are in general antagonists of Activin A, thus, a simple assay to test the potency of the STEM batches is to asses Activin A neutralization in a sensitive cell line such as MPC-11 cell line. The assay development included finding the cell density that was best responsive to the assay. The activin A range that was neutralized by typical STEM lots was used to determine an Activin A IC50, which could be further translated into an Activin A neutralizing units of STEM.

For execution, the assay used Promega CellTiter Glo homogeneous method to determine the number of viable cells in culture based on quantitation of the ATP present, which signaled the presence of metabolically active cells.

Complete media was made using 1:10 STEM in MPC11 feeding media (9 mL feeding media and 1 mL STEM). A sample of cell suspension from the main MPC-11 culture was subjected counting using Nexcelom AO/PI standardized assay. The main culture contained 2.82×10⁴ viable cells/mL, a volume containing 1 million cells (about 30 mL) was extracted and centrifuged 5 minutes at 300 G. The pellet was resuspended in 2.4 mL of media and cells were plated at various concentrations in the test plate and allowed to grow for 4 days in the CO₂ incubator.

TABLE 7
MPC-11 cell distribution in the 96 well plate:

| Row | Cells/well | Cells/row |
|---|---|---|
| A | 20,000 | 60,000 |
| B | 20,000 | 60,000 |
| C | 10,000 | 120,000 |
| D | 10,000 | 120,000 |
| E | 5,000 | 240,000 |
| F | 5,000 | 240,000 |

For each concentration of cells, a range of Activin A between 0.001 ng/ml and 120 ng/ml using a 400 ng/mL Activin A stock solution and serial-diluted at 1:3 dilution progression was tested.

The CellTiter Glo assay was performed according to the product instruction and using the assay template provided by Promega Glomax Explorer microplate reader.

### Results

The best assay sensitivity (maximum luminiscence that is proportional with cell viability) was found using 5,000 cells per well. The other two groups (10,000 and 20,000 cells per well) had luminiscence decreasing proportionally with the cell concentration. This suggest that the high cell density was a limiting factor for the culture condition in general.

TABLE 8
Curve fit data for luminescence - cell viability measurement.

| Reading | Cells /well | Slope | IC50 (ng) | Bottom | Top |
|---|---|---|---|---|---|
| GROUP A | 20,000 | −24.62 | 11.65 | 172316.67 | 245391.67 |
| GROUP B | 10,000 | −24.21 | 12.33 | 274116.67 | 315991.67 |
| GROUP C | 5,000 | −26.01 | 33.52 | 217916.67 | 331226.67 |

It is known that the Follistatin to Activin A neutralizing molar ratio is 2:1. The molecular weight of Activin A is 26.2 kDa, follistatin molecular weight is 31.7 kDa. Thus, the theoretical neutralizing amounts of Follistatin in the assay are presented in TABLE 9.

TABLE 9
Theoretical Follistatin equivalent neutralization concentration
required for Activin IC50 concentration.

| | | | | | Theoretical |
|---|---|---|---|---|---|
| | | | Theoretical neutralizing | | Follistatin |
| | Activin A IC50 | | Follistatin equivalent | | in STEM |
| Reading | (ng) | (nM) | (nM) | (ng) | (ng) |
| GROUP A | 11.65 | 0.44 | 0.88 | 14.10 | 141.0 |
| GROUP B | 12.33 | 0.47 | 0.94 | 14.92 | 149.2 |
| GROUP C | 33.52 | 1.27 | 2.54 | 40.56 | 405.6 |

Comparing the assay result with the quantitative Follistatin determination by ELISA, there was a large discrepancy between its biological activity as shown in the MPC-1 assay and its quantitative determination by ELISA (see TABLE 6).

An important finding is that the Activin A in combination with STEM has a biphasic effect with a pro-stimulatory peak around 3.7 ng/mL, then an inhibitory effect with an IC50 of about 20 ng/mL.

### CONCLUSIONS

The determined equivalent follistatin bioactivity is much larger that the amount determined by quantitative ELISA.

The combination of STEM and low concentration of Activin A had an unexpected stimulative effect on the MPC-11 cells,

The data is suggestive that Follistatin is not the sole component responsible for the biological effect of STEM on the MPC-11 cell system. This finding supports the evaluation of STEM based as a whole rather than individual components.

The finding also suggests that therapeutic doses of STEM could be potentiated by low amounts of Activin A existing in vivo, which could explain the effects observed in previous animal experiments where the relatively low doses of STEM exerted significant biological effects.

Various aspects of the present subject matter are set forth below, in review of, and/or in supplementation to, the embodiments described thus far, with the emphasis here being on the interrelation and interchangeability of the following embodiments. In other words, an emphasis is on the fact that each feature of the embodiments can be combined with each and every other feature unless explicitly stated otherwise or logically implausible. The embodiments described herein are restated and expanded upon in the following paragraphs without explicit reference to the figures.

In many methods, a method of preparing a secretome is described. The method includes the steps of: expanding pluripotent stem cells in culture medium comprising bFGF and Activin A; exposing the pluripotent stem cells to a protein-free media comprising trehalose; harvesting the culture supernatant daily and replacing with additional protein-free media comprising trehalose; combining the harvested culture supernatant to form a combined pool of culture supernatant; homogenizing the combined pool of culture supernatant to form a homogenized combined pool of culture supernatant; and filtering the homogenized combined pool of culture supernatant through a membrane filter to form a filtered homogenized combined pool.

In some embodiments, the pluripotent step cells may be expanded for about 3 days to about 7 days.

In some embodiments, a concentration of trehalose in the protein-free medium is between about 5% and about 25%.

In some embodiments, the culture supernatant is harvested daily and replaced with additional protein-free media comprising trehalose for about 1 day to about 10 days.

In some embodiments, the method further includes the step of reducing a volume of the filtered homogenized combined pool using a tangential flow filtration. In some embodiments, the volume reduction by tangential flow filtration is 50:1 to 100:1. In some embodiments, the tangential flow filtration utilizes a 1 to 5 kDa cutoff membrane.

In many embodiments, a secretome composition obtained by the methods described herein is described.

In some embodiments, the composition comprises at least two myogenic agonists. In some embodiments, the composition comprises at least two myogenic agonists selected from the group consisting of Fetuin A, Follistatin, and Follistatin like-1. In some embodiments, the at least two myogenic agonists may include Follistatin, Follistatin variants, or fragments of Follistatin. In some embodiments, a combined concentration of the at least two myogenic agonists is between about 1 to about 20 pmol/mL. In some embodiments, the at least two myogenic agonists further comprise any one or more of OPN, IGFBP-4, MMP-1, TSP-1, GROa (CXCL1), MMP-10, bIG-H3, hCGb, RGM-B, VEGF, Cripto-1, HGF, BMP-5, bFGF, IGF-2, PDGF-AA, FGF-19, WISP-1, or combinations thereof.

In some embodiments, live cells have been removed from the secretome composition.

In some embodiments, the composition further comprises at least two myogenic antagonists. In some embodiments, the composition has a concentration of the at least two myogenic antagonists below about 1 pmol/mL. In some embodiments, the at least two myogenic antagonists comprises Activin A and TGFb1.

In some embodiments, the ratio of a concentration of the at least two myogenic agonists to the at least two myogenic antagonists is between about 4:1 and about 8:1. In some embodiments, the ratio of a concentration of the at least two myogenic agonists to the at least two myogenic antagonists is between about 5:1 and about 8:1. In some embodiments, the ratio of a concentration of the at least two myogenic agonists to the at least two myogenic antagonists is between about 5:1 and about 200:1.

In many embodiments, a pharmaceutical composition is described that includes the secretome composition described in any of the embodiments described herein, a pharmaceutically acceptable carrier; and a pharmaceutically acceptable preservative.

In some embodiments, the pharmaceutically acceptable carrier is a physiological buffer with a pH of 7.0-7.8.

In some embodiments, the pharmaceutically acceptable preservative is trehalose.

In some embodiments, the pharmaceutical composition is powder preserved by freeze-dry methods.

In some embodiments, the pharmaceutical composition is sterilized by gamma irradiation.

In some embodiments, the pharmaceutical composition is packaged in single dose glass container or ready to use single dose syringe.

In some embodiments, the pharmaceutical composition further comprises a corticosteroid.

In some embodiments, the pharmaceutical composition further comprises an anabolic steroid.

In many embodiments, a method for treating muscles is also described. The method includes administered to a subject a composition described in any of the preceding claims or elsewhere in the application.

In some embodiments, the composition is administered by injection. In some embodiments, the injection is administered intramuscularly, intravenously, or subcutaneously.

In some embodiments, the composition is administered once, daily, weekly, or monthly.

In many embodiments, a method for treating muscles is described. The method includes the steps of administering to a subject a composition comprising: a therapeutically effective amount of a secretome derived from partially differentiated embryonic stem cells, wherein the secretome comprises myogenic agonists and myogenic antagonists in a ratio of at least about 5:1, wherein the myogenic agonists comprise Fetuin A; and a pharmaceutically acceptable carrier.

In some embodiments, the secretome is in a form of a cell conditioned medium obtained by culturing partially differentiated embryonic stem cells in a cell culture medium comprising bFDF and Activin A, and wherein live cells have been removed.

In some embodiments, the secretome comprises Fetuin A in a concentration of between about 5 to about 15 pmol/mL, alternatively between about 1 to about 20 pmol/mL.

In some embodiments, the ratio of myogenic agonists to myogenic antagonists is between about 4:1 and about 8:1, alternatively between about 5:1 and about 7:1, alternatively between about 6:1 and about 7:1, alternatively between about 5:1 and about 50:1, alternatively between about 5:1 and about 100:1, alternatively between about 5:1 and about 150:1, alternatively between about 5:1 and about 200:1.

In some embodiments, the pharmaceutically acceptable carrier is PBS.

In some embodiments, the composition has a concentration of the myogenic antagonists below about 1 pmol/mL. In some embodiments, the myogenic antagonists comprise Activin A. In some embodiments, the myogenic antagonists comprise TGFb1.

In some embodiments, the myogenic agonists further comprise Follistatin, GROa, osteopontin (OPN), IGFBP-4, and/or MMP proteins (such as MMP-10).

In many embodiments, a secretome is described that is derived from embryonic stem cells comprising a mixture of myogenic agonists and myogenic antagonists in a ratio of at least 5:1, wherein the myogenic agonists comprise Fetuin A.

In some embodiments, the secretome is in a form of a cell conditioned medium obtained by culturing partially differentiated embryonic stem cells in a cell culture medium comprising bFDF and Activin A, and wherein live cells have been removed.

In some embodiments, live cells have been removed from the cell conditioned medium.

In some embodiments, the secretome comprises Fetuin A in a concentration of between about 5 to about 15 pmol/mL, alternatively between about 1 to about 20 pmol/mL.

In some embodiments, the ratio of myogenic agonists to myogenic antagonists is between about 4:1 and about 8:1, alternatively between about 5:1 and about 7:1, alternatively between about 6:1 and about 7:1, alternatively between about 5:1 and about 50:1, alternatively between about 5:1 and about 100:1, alternatively between about 5:1 and about 150:1, alternatively between about 5:1 and about 200:1.

In some embodiments, the pharmaceutically acceptable carrier is PBS.

In some embodiments, the myogenic antagonists comprise Activin A. In some embodiments, the myogenic antagonists comprise TGFb1.

In some embodiments, the myogenic agonists further comprise Follistatin, GROa, osteopontin (OPN), IGFBP-4, and/or MMP proteins (such as MMP-10).

In many embodiments, a method for treating muscles is described. The method includes the steps of administering to a subject a composition comprising: a therapeutically effective amount of a secretome derived from partially differentiated embryonic stem cells, wherein the secretome comprises Fetuin A, IGFBP4, Activin A, and Follistatin; and a pharmaceutically acceptable carrier.

In some embodiments, the secretome is in a form of a cell conditioned medium obtained by culturing partially differentiated embryonic stem cells in a cell culture medium comprising bFDF and Activin A, wherein live cells have been removed.

In some embodiments, a concentration of Fetuin A is between about 5 to about 15 pmol/mL, alternatively between about 1 to about 20 pmol/mL.

In some embodiments, the secretome further comprises GROa, osteopontin (OPN), and/or MMP proteins (such as MMP-10).

In many embodiments, a secretome is described that is derived from embryonic stem cells comprising at least two myogenic agonists and at least two myogenic antagonists.

In some embodiments, the secretome has a concentration of the at least two myogenic antagonists below about 1 pmol/mL.

In some embodiments, the at least two myogenic antagonists comprises Activin A and TGFb1.

In some embodiments, the ratio of the at least two myogenic agonists to the at least two myogenic antagonists is between about 4:1 and about 8:1, alternatively between about 5:1 and about 7:1, alternatively between about 6:1 and about 7:1, alternatively between about 5:1 and about 50:1, alternatively between about 5:1 and about 100:1, alternatively between about 5:1 and about 150:1, alternatively between about 5:1 and about 200:1.

In some embodiments, the at least two myogenic agonists comprise Fetuin A, wherein a concentration of Fetuin A is between about 5 to about 15 pmol/mL, alternatively between about 1 to about 20 pmol/mL.

In some embodiments, the at least two myogenic antagonists comprise Activin A. In some embodiments, the at least two myogenic antagonists comprise TGFb1.

In some embodiments, the at least two myogenic agonists further comprise Follistatin, GROa, osteopontin (OPN), IGFBP-4, and/or MMP proteins (such as MMP-10).

In many embodiments, a pharmaceutical composition comprises a secretome derived from embryonic stem cells comprising at least two myogenic agonists and at least two myogenic antagonists; a pharmaceutically acceptable carrier; and a pharmaceutically acceptable preservative.

In some embodiments, the secretome has a concentration of the at least two myogenic antagonists below about 1 pmol/mL.

In some embodiments, the at least two myogenic antagonists comprises Activin A and TGFb1.

In some embodiments, the ratio of the at least two myogenic agonists to the at least two myogenic antagonists is between about 4:1 and about 8:1, alternatively between about 5:1 and about 7:1, alternatively between about 6:1 and about 7:1, alternatively between about 5:1 and about 50:1, alternatively between about 5:1 and about 100:1, alternatively between about 5:1 and about 150:1, alternatively between about 5:1 and about 200:1.

In some embodiments, the at least two myogenic agonists comprise Fetuin A, wherein a concentration of Fetuin A is between about 5 to about 15 pmol/mL, alternatively between about 1 to about 20 pmol/mL.

In some embodiments, the at least two myogenic antagonists comprise Activin A. In some embodiments, the at least two myogenic antagonists comprise TGFb1.

In some embodiments, the at least two myogenic agonists further comprise Follistatin, GROa, osteopontin (OPN), IGFBP-4, and/or MMP proteins (such as MMP-10).

In some embodiments, the pharmaceutically acceptable carrier is a physiological buffer with a pH of 7.0-7.8.

In some embodiments, the pharmaceutically acceptable preservative is trehalose.

In some embodiments, the pharmaceutical composition is powder preserved by freeze-dry methods.

In some embodiments, the pharmaceutical composition is sterilized by gamma irradiation.

In some embodiments, the pharmaceutical composition is packaged in single dose glass container or ready to use single dose syringe.

In some embodiments, the pharmaceutical composition further comprises a corticosteroid.

In some embodiments, the pharmaceutical composition further comprises an anabolic steroid.

In many embodiments, a pharmaceutical composition comprises (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than 10% of total proteins in the composition. Sequences of follistatin protein and variants thereof are listed in FIG. 7.

In some embodiments, the follistatin protein comprises an amino acid sequence selected from the group consisting of SEQ ID NO: 1-13.

In some embodiments, the variant of the follistatin protein has at least 75% sequence identity, or preferably at least 80%, 85%, 90% or 95% sequence identity, to any one of SEQ ID NO:1-13.

In some embodiments, the variant of the follistatin protein comprises a follistatin-N-terminal (FOLN) domain. In some embodiments, the FOLN domain comprises an amino acid sequence selected from the group consisting of SEQ ID NO:14-16, or an amino acid sequence having at least 85% sequence identity to any one of SEQ ID NO:14-16. In some embodiments, the FOLN domain comprises an amino acid sequence selected from the group consisting of SEQ ID NO: 14-16.

In some embodiments, the fetuin A protein comprises an amino acid sequence selected from the group consisting of SEQ ID NO: 17-20.

In some embodiments, the fragment or variant of the fetuin A protein comprises the amino acid sequence of SEQ ID NO:27 or an amino acid sequence having at least 85% sequence identity to SEQ ID NO:27.

In some embodiments, the fragment or variant of the fetuin A protein is a single-chain or two-chain protein.

In some embodiments, the (A) comprises an amino acid sequence selected from the group consisting of SEQ ID NO: 1-13 and the (B) comprises an amino acid sequence selected from the group consisting of SEQ ID NO: 17-20 or is a fragment of any one of SEQ ID NO:17-20 that comprises the fragment of SEQ ID NO:27.

In some embodiments, the total amount of (A) and (B) is greater than 20%, or preferably greater than 25%, 30%, 35% or 40%, of total proteins in the composition.

In some embodiments, the total proteins do not include more than 20% of myogenic antagonist factors, wherein the myogenic antagonist factors comprise IGFBP-3 (insulin like growth factor binding protein 3) and Dkk-3 (dickkopf WNT signaling pathway inhibitor 3). In some embodiments, the myogenic antagonist factors further comprise Periostin, TGFb1, IL-6, FGF-21, TGFb2, TNFb, TGFb3, Activin A, TGFb1 and TGFa.

In some embodiments, the pharmaceutical composition further includes one or more pro-myogenic factors. In some embodiments, the pro-myogenic factors comprise one or more of OPN, IGFBP-4, MMP-1, TSP-1, GROa, MMP-10, bIG-H3, hCGb, RGM-B, VEGF, Cripto-1, HGF, BMP-5, bFGF, IGF-2, PDGF-AA, FGF-19, and WISP-1.

In some embodiments, the total amount of pro-myogenic factors and the total amount of myogenic antagonist factors have a ratio of greater than 3:1, or preferably greater than 4:1, 5:1, 6:1, 7:1, or 8:1.

In many embodiments, a method for improving the muscle mass, strength or function in a patient includes the step of administering to the patient the pharmaceutical composition of any of the embodiments described herein.

In some embodiments, the administration is intramuscular, intravenous, or subcutaneous.

In some embodiments, the patient suffers from muscle injury, muscle atrophy, muscle dysfunction, or muscle fibrosis.

In many embodiments, a method of preparing a secretome composition, including the steps of: culturing stem cells in a protein-free media comprising trehalose at a concentration of from 5% to 25% w/v; harvesting supernatant from the sample and replacing the supernatant with a fresh protein-free media comprising trehalose at a concentration of from 5% to 25% w/v; and combining the harvested supernatant to prepare the secretome composition.

In many embodiments, a pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than 10% of total proteins in the composition.

In some embodiments, the follistatin protein comprises an amino acid sequence selected from the group consisting of SEQ ID NO: 1-13. In some embodiments, the variant of the follistatin protein has at least 75% sequence identity to any one of SEQ ID NO:1-13. In some embodiments, the variant of the follistatin protein comprises a follistatin-N-terminal (FOLN) domain. In some embodiments, the FOLN domain comprises an amino acid sequence selected from the group consisting of SEQ ID NO: 14-16, or an amino acid sequence having at least 85% sequence identity to any one of SEQ ID NO:14-16. In some embodiments, the FOLN domain comprises an amino acid sequence selected from the group consisting of SEQ ID NO:14-16.

In some embodiments, the fetuin A protein comprises an amino acid sequence selected from the group consisting of SEQ ID NO: 17-20. In some embodiments, the fragment or variant of the fetuin A protein comprises the amino acid sequence of SEQ ID NO:27 or an amino acid sequence having at least 85% sequence identity to SEQ ID NO:27. In some embodiments, the fragment or variant of the fetuin A protein is a single-chain or two-chain protein.

In some embodiments, the (A) comprises an amino acid sequence selected from the group consisting of SEQ ID NO: 1-13 and the (B) comprises an amino acid sequence selected from the group consisting of SEQ ID NO: 17-20 or is a fragment of any one of SEQ ID NO: 17-20 that comprises the fragment of SEQ ID NO:27.

In some embodiments, the total amount of (A) and (B) is greater than 20% of total proteins in the composition.

In some embodiments, the total proteins do not include more than 20% of myogenic antagonist factors, wherein the myogenic antagonist factors comprise IGFBP-3 (insulin like growth factor binding protein 3) and Dkk-3 (dickkopf WNT signaling pathway inhibitor 3). In some embodiments, the myogenic antagonist factors further comprise Periostin, TGFb1, IL-6, FGF-21, TGFb2, TNFb, TGFb3, Activin A, TGFb1 and TGFa.

In some embodiments, the composition further includes one or more pro-myogenic factors. In some embodiments, the pro-myogenic factors comprise one or more of OPN, IGFBP-4, MMP-1, TSP-1, GROa, MMP-10, bIG-H3, hCGb, RGM-B, VEGF, Cripto-1, HGF, BMP-5, bFGF, IGF-2, PDGF-AA, FGF-19, and WISP-1.

In some embodiments, the total amount of pro-myogenic factors and the total amount of myogenic antagonist factors have a ratio of greater than 3:1.

In many embodiments, a method for improving the muscle mass, strength, or function in a patient includes the steps of: administering to the patient a pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than 10% of total proteins in the composition.

In some embodiments, the administration is intramuscular, intravenous, or subcutaneous.

In some embodiments, the patient suffers from muscle injury, muscle atrophy, muscle dysfunction, or muscle fibrosis.

In many embodiments, a method of preparing a secretome composition includes the steps of: culturing stem cells in a protein-free media comprising trehalose at a concentration of from 5% to 25% w/v; harvesting supernatant from the sample and replacing the supernatant with a fresh protein-free media comprising trehalose at a concentration of from 5% to 25% w/v; and combining the harvested supernatant to prepare the secretome composition.

In some embodiments, the method further includes the step of reducing a volume of the filtered homogenized combined pool using a tangential flow filtration. In some embodiments, the volume reduction by tangential flow filtration is 50:1 to 100:1. In some embodiments, the tangential flow filtration utilizes a 1 to 5 kDa cutoff membrane.

In some embodiments, the method further includes the step of removing live cells from the harvested supernatant.

In many embodiments, a method of preparing a secretome composition, the method comprising the steps of: expanding pluripotent stem cells in culture medium comprising bFGF and Activin A for about 3 days to about 7 days; exposing the pluripotent stem cells to a protein-free media comprising trehalose, wherein the trehalose concentration is between 5% and 25%; harvesting the culture supernatant daily and replacing with additional protein-free media comprising trehalose for about 1 day to about 10 days; combining the harvested culture supernatant to form a combined pool of culture supernatant; homogenizing the combined pool of culture supernatant to form a homogenized combined pool of culture supernatant; and filtering the homogenized combined pool of culture supernatant through a membrane filter to form a filtered homogenized combined pool.

In some embodiments, the method further includes the step of reducing a volume of the filtered homogenized combined pool using a tangential flow filtration. In some embodiments, the volume reduction by tangential flow filtration is 50:1 to 100:1. In some embodiments, the tangential flow filtration utilizes a 1 to 5 kDa cutoff membrane.

In some embodiments, further comprising the step of removing live cells from the filtered homogenized combined pool.

In some embodiments, a secretome composition comprising: at least two myogenic agonists selected from the group consisting of Fetuin A, Follistatin, and Follistatin like-1, wherein a combined concentration of the at least two myogenic agonists is between about 1 to about 20 pmol/mL.

In some embodiments, the at least two myogenic agonists further comprise OPN, IGFBP-4, MMP-1, TSP-1, GROa (CXCL1), MMP-10, bIG-H3, hCGb, RGM-B, VEGF, Cripto-1, HGF, BMP-5, bFGF, IGF-2, PDGF-AA, FGF-19, WISP-1, or combinations thereof.

In some embodiments, the composition further comprises at least two myogenic antagonists. In some embodiments, the composition has a concentration of the at least two myogenic antagonists below about 1 pmol/mL. In some embodiments, the at least two myogenic antagonists comprise Activin A and TGFb1.

In some embodiments, a ratio of a concentration of the at least two myogenic agonists to the at least two myogenic antagonists is between about 4:1 and about 8:1. In some embodiments, a ratio of a concentration of the at least two myogenic agonists to the at least two myogenic antagonists is between about 5:1 and about 8:1. In some embodiments, a ratio of a concentration of the at least two myogenic agonists to the at least two myogenic antagonists is between about 5:1 and about 200:1.

In many embodiments, a pharmaceutical composition, comprising: at least two myogenic agonists selected from the group consisting of Fetuin A, Follistatin, and Follistatin like-1, wherein a combined concentration of the at least two myogenic agonists is between about 1 to about 20 pmol/mL; a pharmaceutically acceptable carrier; and a pharmaceutically acceptable preservative.

In some embodiments, the pharmaceutically acceptable carrier is a physiological buffer with a pH of 7.0-7.8.

In some embodiments, the pharmaceutically acceptable preservative is trehalose.

In some embodiments, the pharmaceutical composition is powder preserved by freeze-dry methods.

In some embodiments, the pharmaceutical composition is sterilized by gamma irradiation.

In some embodiments, the pharmaceutical composition is packaged in single dose glass container or ready to use single dose syringe.

In some embodiments, the pharmaceutical composition further comprises a corticosteroid.

In some embodiments, the pharmaceutical composition further comprises an anabolic steroid.

In some embodiments, a method for treating muscles, the method comprising the step of: administering to a subject a composition comprising at least two myogenic agonists selected from the group consisting of Fetuin A, Follistatin, and Follistatin like-1, wherein a combined concentration of the at least two myogenic agonists is between about 1 to about 20 pmol/mL; a pharmaceutically acceptable carrier; and a pharmaceutically acceptable preservative. In some embodiments, the composition is administered by injection.

In some embodiments, the injection is administered intramuscularly, intravenously, or subcutaneously.

In some embodiments, the composition is administered once, daily, weekly, or monthly.

In any of the above embodiments, the compositions may include Follistatin, Follistatin variants, or fragments of Follistatin. In any of the above embodiments, the compositions may include Fetuin A, Fetuin A variants, or fragments of Fetuin A.

### Clauses

Exemplary embodiments are set out in the following numbered clauses.

Clause 1. A pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than 10% of total proteins in the composition.

Clause 2. The pharmaceutical composition of clause 1, wherein the follistatin protein comprises an amino acid sequence selected from the group consisting of SEQ ID NO:1-13.

Clause 3. The pharmaceutical composition of clause 2, wherein the variant of the follistatin protein has at least 75% sequence identity to any one of SEQ ID NO:1-13.

Clause 4. The pharmaceutical composition of clause 3, wherein the variant of the follistatin protein comprises a follistatin-N-terminal (FOLN) domain.

Clause 5. The pharmaceutical composition of clause 4, wherein the FOLN domain comprises an amino acid sequence selected from the group consisting of SEQ ID NO:14-16, or an amino acid sequence having at least 85% sequence identity to any one of SEQ ID NO: 14-16.

Clause 6. The pharmaceutical composition of clause 4, wherein the FOLN domain comprises an amino acid sequence selected from the group consisting of SEQ ID NO: 14-16.

Clause 7. The pharmaceutical composition of clause 1, wherein the fetuin A protein comprises an amino acid sequence selected from the group consisting of SEQ ID NO:17-20.

Clause 8. The pharmaceutical composition of clause 7, wherein the fragment or variant of the fetuin A protein comprises the amino acid sequence of SEQ ID NO:27 or an amino acid sequence having at least 85% sequence identity to SEQ ID NO:27.

Clause 9. The pharmaceutical composition of clause 7, wherein the fragment or variant of the fetuin A protein is a single-chain or two-chain protein.

Clause 10. The pharmaceutical composition of clause 1, wherein the (A) comprises an amino acid sequence selected from the group consisting of SEQ ID NO:1-13 and the (B) comprises an amino acid sequence selected from the group consisting of SEQ ID NO:17-20 or is a fragment of any one of SEQ ID NO:17-20 that comprises the fragment of SEQ ID NO:27.

Clause 11. The pharmaceutical composition of any one of clause 1, wherein the total amount of (A) and (B) is greater than 20% of total proteins in the composition.

Clause 12. The pharmaceutical composition of clause 1, wherein the total proteins do not include more than 20% of myogenic antagonist factors, wherein the myogenic antagonist factors comprise IGFBP-3 (insulin like growth factor binding protein 3) and Dkk-3 (dickkopf WNT signaling pathway inhibitor 3).

Clause 13. The pharmaceutical composition of 12, wherein the myogenic antagonist factors further comprise Periostin, TGFb1, IL-6, FGF-21, TGFb2, TNFb, TGFb3, Activin A, TGFb1 and TGFa.

Clause 14. The pharmaceutical composition of clause 1, further comprising one or more pro-myogenic factors.

Clause 15. The pharmaceutical composition of clause 14, wherein the pro-myogenic factors comprise one or more of OPN, IGFBP-4, MMP-1, TSP-1, GROa, MMP-10, bIG-H3, hCGb, RGM-B, VEGF, Cripto-1, HGF, BMP-5, bFGF, IGF-2, PDGF-AA, FGF-19, and WISP-1.

Clause 16. The pharmaceutical composition of clause 1, wherein the total amount of pro-myogenic factors and the total amount of myogenic antagonist factors have a ratio of greater than 3:1.

Clause 17. A method for improving the muscle mass, strength, or function in a patient, comprising the step of:

administering to the patient a pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than 10% of total proteins in the composition.

Clause 18. The method of clause 17, wherein the administration is intramuscular, intravenous, or subcutaneous.

Clause 19. The method of clause 17, wherein the patient suffers from muscle injury, muscle atrophy, muscle dysfunction, or muscle fibrosis.

Clause 20. A method of preparing a secretome composition, comprising the steps of:
culturing stem cells in a protein-free media comprising trehalose at a concentration of from 5% to 25% w/v;
harvesting supernatant from the sample and replacing the supernatant with a fresh protein-free media comprising trehalose at a concentration of from 5% to 25% w/v; and
combining the harvested supernatant to prepare the secretome composition.

Clause 21. The method of clause 20, further comprising the step of reducing a volume of the filtered homogenized combined pool using a tangential flow filtration.

Clause 22. The method of clause 21, wherein the volume reduction by tangential flow filtration is 50:1 to 100:1.

Clause 23. The method of clause 22, wherein the tangential flow filtration utilizes a 1 to 5 kDa cutoff membrane.

Clause 24. The method of clause 20, further comprising the step of removing live cells from the harvested supernatant.

Clause 25. A method of preparing a secretome composition, the method comprising the steps of:
expanding pluripotent stem cells in culture medium comprising bFGF and Activin A for about 3 days to about 7 days;
exposing the pluripotent stem cells to a protein-free media comprising trehalose, wherein the trehalose concentration is between 5% and 25%;
harvesting the culture supernatant daily and replacing with additional protein-free media comprising trehalose for about 1 day to about 10 days;
combining the harvested culture supernatant to form a combined pool of culture supernatant;
homogenizing the combined pool of culture supernatant to form a homogenized combined pool of culture supernatant; and
filtering the homogenized combined pool of culture supernatant through a membrane filter to form a filtered homogenized combined pool.

Clause 26. The method of clause 25, further comprising the step of reducing a volume of the filtered homogenized combined pool using a tangential flow filtration.

Clause 27. The method of clause 26, wherein the volume reduction by tangential flow filtration is 50:1 to 100:1.

Clause 28. The method of clause 26, wherein the tangential flow filtration utilizes a 1 to 5 kDa cutoff membrane.

Clause 29. The method of clause 25, further comprising the step of removing live cells from the filtered homogenized combined pool.

Clause 30. A secretome composition comprising:
at least two myogenic agonists selected from the group consisting of Fetuin A, Follistatin, and Follistatin like-1,
wherein a combined concentration of the at least two myogenic agonists is between about 1 to about 20 pmol/mL.

Clause 31. The composition of clause 30, wherein the at least two myogenic agonists further comprise OPN, IGFBP-4, MMP-1, TSP-1, GROa (CXCL1), MMP-10, bIG-H3, hCGb, RGM-B, VEGF, Cripto-1, HGF, BMP-5, bFGF, IGF-2, PDGF-AA, FGF-19, WISP-1, or combinations thereof.

Clause 32. The composition of clause 30, wherein the composition further comprises at least two myogenic antagonists.

Clause 33. The composition of clause 32, wherein the composition has a concentration of the at least two myogenic antagonists below about 1 pmol/mL.

Clause 34. The composition of clause 32, wherein the at least two myogenic antagonists comprise Activin A and TGFb1.

Clause 35. The composition of clause 32, wherein a ratio of a concentration of the at least two myogenic agonists to the at least two myogenic antagonists is between about 4:1 and about 8:1.

Clause 36. The composition of clause 32, wherein a ratio of a concentration of the at least two myogenic agonists to the at least two myogenic antagonists is between about 5:1 and about 8:1.

Clause 37. The composition of clause 32, wherein a ratio of a concentration of the at least two myogenic agonists to the at least two myogenic antagonists is between about 5:1 and about 200:1.

Clause 38. A pharmaceutical composition, comprising:
at least two myogenic agonists selected from the group consisting of Fetuin A, Follistatin, and Follistatin like-1, wherein a combined concentration of the at least two myogenic agonists is between about 1 to about 20 pmol/mL;
a pharmaceutically acceptable carrier; and
a pharmaceutically acceptable preservative.

Clause 39. The composition of clause 38, wherein the pharmaceutically acceptable carrier is a physiological buffer with a pH of 7.0-7.8.

Clause 40. The composition of clause 38, wherein the pharmaceutically acceptable preservative is trehalose.

Clause 41. The composition of clause 38, wherein the pharmaceutical composition is powder preserved by freeze-dry methods.

Clause 42. The composition of clause 38, wherein the pharmaceutical composition is sterilized by gamma irradiation.

Clause 43. The composition of clause 38, wherein the pharmaceutical composition is packaged in single dose glass container or ready to use single dose syringe.

Clause 44. The composition of clause 38, wherein the pharmaceutical composition further comprises a corticosteroid.

Clause 45. The composition of clause 38, wherein the pharmaceutical composition further comprises an anabolic steroid.

Clause 46. A method for treating muscles, the method comprising the step of:
administering to a subject a composition comprising at least two myogenic agonists selected from the group consisting of Fetuin A, Follistatin, and Follistatin like-1, wherein a combined concentration of the at least two myogenic agonists is between about 1 to about 20 pmol/mL; a pharmaceutically acceptable carrier; and a pharmaceutically acceptable preservative.

Clause 47. The method of clause 46, wherein the composition is administered by injection.

Clause 48. The method of clause 47, wherein the injection is administered intramuscularly, intravenously, or subcutaneously.

Clause 49. The method of clause 47, wherein the composition is administered once, daily, weekly, or monthly.

### REFERENCES

1. Degens, H., and Alway, S. E. (2006) Control of muscle size during disuse, disease, and aging. Int J Sports Med 27, 94-99
2. Rcidy, P. T., Lindsay, C. C., Mckenzie, A. I., Fry, C. S., Supiano, M. A., Marcus, R. L., LaStayo, P. C., and Drummond, M. J. (2018) Aging-related effects of bed rest followed by eccentric exercise rehabilitation on skeletal muscle macrophages and insulin sensitivity. Exp Gerontol 107, 37-49
3. Suetta, C., Frandsen, U., Jensen, L., Jensen, M. M., Jespersen, J. G., Hvid, L. G., Bayer, M., Petersson, S. J., Schroder, H. D., Andersen, J. L., Heinemeier, K. M., Aagaard, P., Schjerling, P., and Kjaer, M. (2012) Aging affects the transcriptional regulation of human skeletal muscle disuse atrophy. PloS one 7, c51238
4. Cangelosi, D., Resaz, R., Petretto, A., Segalerba, D., Ognibene, M., Raggi, F., Mastracci, L., Grillo, F., Bosco, M. C., Varesio, L., Sica, A., Colombo, I., and Eva, A. (2019) A Proteomic Analysis of GSD-la in Mouse Livers: Evidence for Metabolic Reprogramming, Inflammation, and Macrophage Polarization. J Proteome Res 18, 2965-2978
5. Li, C. I., Li, T. C., Lin, W. Y., Liu, C. S., Hsu, C. C., Hsiung, C. A., Chen, C. Y., Huang, K. C., Wu, C. H., Wang, C. Y., Lin, C. C., Sarcopenia, and Translational Aging Research in Taiwan, T. (2015) Combined association of chronic disease and low skeletal muscle mass with physical performance in older adults in the Sarcopenia and Translational Aging Research in Taiwan (START) study. BMC Geriatr 15, 11
6. Evans, W. J. (2010) Skeletal muscle loss: cachexia, sarcopenia, and inactivity. Am J Clin Nutr 91, 1123S-1127S
7. Evans, W. J. (2015) Sarcopenia Should Reflect the Contribution of Age-Associated Changes in Skeletal Muscle to Risk of Morbidity and Mortality in Elderly People. J Am Med Dir Assoc 16, 546-547
8. Kiriya, Y., Toshiaki, N., Shibasaki, I., Ogata, K., Ogawa, H., Takci, Y., Tezuka, M., Seki, M., Kato, T., Lefor, A. K., and Fukuda, H. (2020) Sarcopenia assessed by the quantity and quality of skeletal muscle is a prognostic factor for patients undergoing cardiac surgery. Surg Today 50, 895-904
9. Miller, M. S., Callahan, D. M., and Toth, M. J. (2014) Skeletal muscle myofilament adaptations to aging, disease, and disuse and their effects on whole muscle performance in older adult humans. Front Physiol 5, 369
10. Wall, B. T., Dirks, M. L., and van Loon, L. J. (2013) Skeletal muscle atrophy during short-term disuse: implications for age-related sarcopenia. Ageing research reviews 12, 898-906
11. Haus, J. M., Carrithers, J. A., Trappe, S. W., and Trappe, T. A. (2007) Collagen, cross-linking, and advanced glycation end products in aging human skeletal muscle. J Appl Physiol (1985) 103, 2068-2076
12. Kovanen, V., Suominen, H., and Peltonen, L. (1987) Effects of aging and life-long physical training on collagen in slow and fast skeletal muscle in rats. A morphometric and immuno-histochemical study. Cell Tissue Res 248, 247-255
13. Alway, S. E., Pereira, S. L., Edens, N. K., Hao, Y., and Bennett, B. T. (2013) beta-Hydroxy-beta-methylbutyrate (HMB) enhances the proliferation of satellite cells in fast muscles of aged rats during recovery from disuse atrophy. Exp Gerontol 48, 973-984
14. Lacraz, G., Rouleau, A. J., Couture, V., Sollrald, T., Drouin, G., Veillette, N., Grandbois, M., and Grenier, G. (2015) Increased Stiffness in Aged Skeletal Muscle Impairs Muscle Progenitor Cell Proliferative Activity. PLOS One 10, c0136217
15. Mahdy, M. A. A. (2019) Skeletal muscle fibrosis: an overview. Cell Tissue Res 375, 575-588
16. Yoshimura, A., Sakamoto, J., Honda, Y., Kataoka, H., Nakano, J., and Okita, M. (2017) Cyclic muscle twitch contraction inhibits immobilization-induced muscle contracture and fibrosis in rats. Connect Tissue Res 58, 487-495
17. Miller, T. A., Lesniewski, L. A., Muller-Delp, J. M., Majors, A. K., Scalise, D., and Delp, M. D. (2001) Hindlimb unloading induces a collagen isoform shift in the soleus muscle of the rat. American journal of physiology. Regulatory, integrative and comparative physiology 281, R1710-1717
18. Etienne, J., Liu, C., Skinner, C. M., Conboy, M. J., and Conboy, I. M. (2020) Skeletal muscle as an experimental model of choice to study tissue aging and rejuvenation. Skelet Muscle 10
19. Yamakawa, H., Kusumoto, D., Hashimoto, H., and Yuasa, S. (2020) Stem Cell Aging in Skeletal Muscle Regeneration and Disease. Int J Mol Sci 21
20. Brack, A. S., Conboy, M. J., Roy, S., Lec, M., Kuo, C. J., Keller, C., and Rando, T. A. (2007) Increased Wnt signaling during aging alters muscle stem cell fate and increases fibrosis. Science 317, 807-810
21. Chakkalakal, J. V., Jones, K. M., Basson, M. A., and Brack, A. S. (2012) The aged niche disrupts muscle stem cell quiescence. Nature 490, 355-360
22. Wang, Y., Wehling-Henricks, M., Welc, S. S., Fisher, A. L., Zuo, Q., and Tidball, J. G. (2019) Aging of the immune system causes reductions in muscle stem cell populations, promotes their shift to a fibrogenic phenotype, and modulates sarcopenia. FASEB J 33, 1415-1427
23. Wang, Y., Wehling-Henricks, M., Samengo, G., and Tidball, J. G. (2015) Increases of M2a macrophages and fibrosis in aging muscle are influenced by bone marrow aging and negatively regulated by muscle-derived nitric oxide. Aging Cell 14, 678-688
24. Tidball, J. G. (2017) Regulation of muscle growth and regeneration by the immune system. Nat Rev Immunol 17, 165-178
25. Reidy, P. T., Mckenzie, A. I., Mahmassani, Z. S., Petrocelli, J. J., Nelson, D. B., Lindsay, C. C., Gardner, J. E., Morrow, V. R., Keefe, A. C., Huffaker, T. B., Stoddard, G. J., Kardon, G., O'Connell, R. M., and Drummond, M. J. (2019) Aging impairs mouse skeletal muscle macrophage polarization and muscle-specific abundance during recovery from disuse. American journal of physiology. Endocrinology and metabolism 317, E85-E98
26. Tidball, J. G., and Wehling-Henricks, M. (2007) Macrophages promote muscle membrane repair and muscle fibre growth and regeneration during modified muscle loading in mice in vivo. The Journal of physiology 578, 327-336
27. Summan, M., Warren, G. L., Mercer, R. R., Chapman, R., Hulderman, T., Van Rooijen, N., and Simconova, P. P. (2006) Macrophages and skeletal muscle regeneration: a clodronate-containing liposome depletion study. American journal of physiology. Regulatory, integrative and comparative physiology 290, R1488-1495
28. Ohira, T., Wang, X. D., Ito, T., Kawano, F., Goto, K., Izawa, T., Ohno, H., Kizaki, T., and Ohira, Y. (2015) Macrophage deficiency in osteopetrotic (op/op) mice inhibits activation of satellite cells and prevents hypertrophy in single soleus fibers. American journal of physiology. Cell physiology 308, C848-855
29. Melton, D. W., Roberts, A. C., Wang, H., Sarwar, Z., Wetzel, M. D., Wells, J. T., Porter, L., Berton, M. T., McManus, L. M., and Shireman, P. K. (2016) Absence of CCR2 results in an inflammaging environment in young mice with age-independent impairments in muscle regeneration. Journal of leukocyte biology 100, 1011-1025
30. Segawa, M., Fukada, S., Yamamoto, Y., Yahagi, H., Kancmatsu, M., Sato, M., Ito, T., Uczumi, A., Hayashi, S., Miyagoc-Suzuki, Y., Takeda, S., Tsujikawa, K., and Yamamoto, H. (2008) Suppression of macrophage functions impairs skeletal muscle regeneration with severe fibrosis. Experimental cell research 314, 3232-3244
31. Alway, S. E., Bennett, B. T., Wilson, J. C., Edens, N. K., and Percira, S. L. (2014) Epigallocatechin-3-gallate improves plantaris muscle recovery after disuse in aged rats. Exp Gerontol 50, 82-94
32. Bennett, B. T., Mohamed, J. S., and Alway, S. E. (2013) Effects of resveratrol on the recovery of muscle mass following disuse in the plantaris muscle of aged rats. PloS one 8, c83518
33. Brooks, M. J., Hajira, A., Mohamed, J. S., and Alway, S. E. (2018) Voluntary wheel running increases satellite cell abundance and improves recovery from disuse in gastrocnemius muscles from mice. J Appl Physiol (1985) 124, 1616-1628
34. Alway, S. E., Bennett, B. T., Wilson, J. C., Sperringer, J., Mohamed, J. S., Edens, N. K., and Pereira, S. L. (2015) Green tea extract attenuates muscle loss and improves muscle function during disuse, but fails to improve muscle recovery following unloading in aged rats. J Appl Physiol (1985) 118, 319-330
35. Chen, K. D., and Alway, S. E. (2001) Clenbuterol reduces soleus muscle fatigue during disuse in aged rats. Muscle Nerve 24, 211-222
36. Arentson-Lantz, E. J., Fiebig, K. N., Anderson-Catania, K. J., Deer, R. R., Wacher, A., Fry, C. S., Lamon, S., and Paddon-Jones, D. (2020) Countering disuse atrophy in older adults with low-volume leucine supplementation. J Appl Physiol (1985) 128, 967-977
37. Morey-Holton, E. R., and Globus, R. K. (2002) Hindlimb unloading rodent model: technical aspects. J Appl Physiol (1985) 92, 1367-1377.
38. Dumont, N. A., and Frenette, J. (2013) Macrophage colony-stimulating factor-induced macrophage differentiation promotes regrowth in atrophied skeletal muscles and C2C12 myotubes. the American Journal of Pathology 182, 505-515
39. Smith, L. R., and Barton, E. R. (2014) SMASH-semi-automatic muscle analysis using segmentation of histology: a MATLAB application. Skeletal muscle 4, 21
40. Kraemer, W. J., and Spicring, B. A. (2006) Skeletal muscle physiology: Plasticity and responses to exercise. Horm Res 66, 2-16
41. Bell, K. E., von Allmen, M. T., Devries, M. C., and Phillips, S. M. (2016) Muscle Disuse as a Pivotal Problem in Sarcopenia-Related Muscle Loss and Dysfunction. J Frality Aging 5, 33-41
42. Miller, B. F., Bachr, L. M., Musci, R. V., Reid, J. J., Peclor, F. F., 3rd, Hamilton, K. L., and Bodine, S. C. (2019) Muscle-specific changes in protein synthesis with aging and reloading after disuse atrophy. Journal of cachexia, sarcopenia and muscle 10, 1195-1209
43. Bachr, L. M., West, D. W., Marcotte, G., Marshall, A. G., De Sousa, L. G., Baar, K., and Bodine, S. C. (2016) Age-related deficits in skeletal muscle recovery following disuse are associated with neuromuscular junction instability and ER stress, not impaired protein synthesis. Aging (Albany NY) 8, 127-146
44. Choi, S. J., Harii, K., Asato, H., and Ueda, K. (1996) Aging effects in skeletal muscle recovery after reinnervation. Scand J Plast Reconstr Surg Hand Surg 30, 89-98
45. Hvid, L. G., Suetta, C., Nielsen, J. H., Jensen, M. M., Frandsen, U., Ortenblad, N., Kjaer, M., and Aagaard, P. (2014) Aging impairs the recovery in mechanical muscle function following 4 days of disuse. Experimental gerontology 52, 1-8
46. Barbe, C., Kalista, S., Loumaye, A., Ritvos, O., Lause, P., Ferracin, B., and Thissen, J. P. (2015) Role of IGF-I in follistatin-induced skeletal muscle hypertrophy. Am J Physiol Endocrinol Metab 309, E557-567
47. Zhu, J., Li, Y., Lu, A., Gharaibch, B., Ma, J., Kobayashi, T., Quintero, A. J., and Huard, J. (2011) Follistatin improves skeletal muscle healing after injury and disease through an interaction with muscle regeneration, angiogenesis, and fibrosis. Am J Pathol 179, 915-930
48. Jennische, E., and Hall, C. M. (2000) Expression and localisation of IGF-binding protein mRNAs in regenerating rat skeletal muscle. APMIS 108, 747-755
49. Schertzer, J. D., Gehrig, S. M., Ryall, J. G., and Lynch, G. S. (2007) Modulation of insulin-like growth factor (IGF)-I and IGF-binding protein interactions enhances skeletal muscle regeneration and ameliorates the dystrophic pathology in mdx mice. Am J Pathol 171, 1180-1188
50. Swiderski, K., Martins, K. J., Chec, A., Tricu, J., Naim, T., Gehrig, S. M., Baum, D. M., Brenmochl, J., Chau, L., Koopman, R., Gregorevic, P., Metzger, F., Hoeflich, A., and Lynch, G. S. (2016) Skeletal muscle-specific overexpression of IGFBP-2 promotes a slower muscle phenotype in healthy but not dystrophic mdx mice and does not affect the dystrophic pathology. Growth Horm IGF Res 30-31, 1-10
51. Calabresi, C., Arosio, B., Galimberti, L., Scanziani, E., Bergottini, R., Annoni, G., and Vergani, C. (2007) Natural aging, expression of fibrosis-related genes and collagen deposition in rat lung. Exp Gerontol 42, 1003-1011
52. Rodrigues, C. J., Rodrigues Junior, A. J., and Bohm, G. M. (1996) Effects of aging on muscle fibers and collagen content of the diaphragm: a comparison with the rectus abdominis muscle. Gerontology 42, 218-228
53. Tidball, J. G., Flores, I., Welc, S. S., Wehling-Henricks, M., and Ochi, E. (2020) Aging of the immune system and impaired muscle regeneration: A failure of immunomodulation of adult myogenesis. Experimental gerontology 145, 111200
54. Cantini, M., Massimino, M. L., Bruson, A., Catani, C., Dalla Libera, L., and Carraro, U. (1994) Macrophages regulate proliferation and differentiation of satellite cells. Biochem Biophys Res Commun 202, 1688-1696
55. Dort, J., Fabre, P., Molina, T., and Dumont, N. A. (2019) Macrophages Are Key Regulators of Stem Cells during Skeletal Muscle Regeneration and Diseases. Stem Cells Int 2019, 4761427
56. Wynn, T. A., and Vannella, K. M. (2016) Macrophages in Tissue Repair, Regeneration, and Fibrosis. Immunity 44, 450-462
57. Tonkin, J., Temmerman, L., Sampson, R. D., Gallego-Colon, E., Barberi, L., Bilbao, D., Schneider, M. D., Musaro, A., and Rosenthal, N. (2015) Monocyte/Macrophage-derived IGF-1 Orchestrates Murine Skeletal Muscle Regeneration and Modulates Autocrine Polarization. Mol Ther 23, 1189-1200
58. Cui, C. Y., Driscoll, R. K., Piao, Y., Chia, C. W., Gorospe, M., and Ferrucci, L. (2019) Skewed macrophage polarization in aging skeletal muscle. Aging Cell 18, e13032
59. Martins, L., Gallo, C. C., Honda, T. S. B., Alves, P. T., Stilhano, R. S., Rosa, D. S., Koh, T. J., and Han, S. W. (2020) Skeletal muscle healing by M1-like macrophages produced by transient expression of exogenous GM-CSF. Stem Cell Res Ther 11, 473
60. Novak, M. L., Weinheimer-Haus, E. M., and Koh, T. J. (2014) Macrophage activation and skeletal muscle healing following traumatic injury. J Pathol 232, 344-355
61. Hammers, D. W., Rybalko, V., Merscham-Banda, M., Hsieh, P. L., Suggs, L. J., and Farrar, R. P. (2015) Anti-inflammatory macrophages improve skeletal muscle recovery from ischemia/reperfusion. J Appl Physiol (1985), jap 00313 02014
62. McKenna, C. F., and Fry, C. S. (2017) Altered satellite cell dynamics accompany skeletal muscle atrophy during chronic illness, disuse, and aging. Curr Opin Clin Nutr Metab Care 20, 447-452
63. Snijders, T., Verdijk, L. B., and van Loon, L. J. (2009) The impact of sarcopenia and exercise training on skeletal muscle satellite cells. Ageing research reviews 8, 328-338
64. Keefe, A. C., Lawson, J. A., Flygare, S. D., Fox, Z. D., Colasanto, M. P., Mathew, S. J., Yandell, M., and Kardon, G. (2015) Muscle stem cells contribute to myofibres in sedentary adult mice. Nat Commun 6, 7087
65. Bonen, A., Tan, M. H., and Watson-Wright, W. M. (1981) Insulin binding and glucose uptake differences in rodent skeletal muscles. Diabetes 30, 702-704
66. Henriksen, E. J., Bourey, R. E., Rodnick, K. J., Koranyi, L., Permutt, M. A., and Holloszy, J. O. (1990) Glucose transporter protein content and glucose transport capacity in rat skeletal muscles. Am J Physiol 259, E593-598
67. Song, X. M., Ryder, J. W., Kawano, Y., Chibalin, A. V., Krook, A., and Zierath, J. R. (1999) Muscle fiber type specificity in insulin signal transduction. Am J Physiol 277, R1690-1696
68. Reidy, P. T., Mckenzie, A. I., Mahmassani, Z. S., Petrocelli, J. J., Nelson, D. B., Lindsay, C. C., Gardner, J. E., Morrow, V. R., Keefe, A. C., Huffaker, T. B., Stoddard, G. J., Kardon, G., O'Connell, R. M., and Drummond, M. J. (2019) Aging impairs mouse skeletal muscle macrophage polarization and muscle-specific abundance during recovery from disuse. American journal of physiology. Endocrinology and metabolism

What is claimed is the following:

1. A pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than about 10% of total proteins in the composition.

2. The pharmaceutical composition of claim 1, wherein the follistatin protein comprises an amino acid sequence selected from the group consisting of SEQ ID NOS: 1-13.

3. The pharmaceutical composition of claim 2, wherein the variant of the follistatin protein has at least 75% sequence identity to any one of SEQ ID NOS: 1-13.

4. The pharmaceutical composition of claim 3, wherein the variant of the follistatin protein comprises a follistatin-N-terminal (FOLN) domain.

5. The pharmaceutical composition of claim 4, wherein the FOLN domain comprises an amino acid sequence selected from the group consisting of SEQ ID NOS: 14-16, or an amino acid sequence having at least 85% sequence identity to any one of SEQ ID NOS: 14-16.

6. The pharmaceutical composition of claim 4, wherein the FOLN domain comprises an amino acid sequence selected from the group consisting of SEQ ID NOS: 14-16.

7. The pharmaceutical composition of claim 1, wherein the fetuin A protein comprises an amino acid sequence selected from the group consisting of SEQ ID NOS: 17-20.

8. The pharmaceutical composition of claim 7, wherein the fragment or variant of the fetuin A protein comprises the amino acid sequence of SEQ ID NO:27 or an amino acid sequence having at least 85% sequence identity to SEQ ID NO:27.

9. The pharmaceutical composition of claim 7, wherein the fragment or variant of the fetuin A protein is a single-chain or two-chain protein.

10. The pharmaceutical composition of claim 1, wherein the (A) comprises an amino acid sequence selected from the group consisting of SEQ ID NOS: 1-13 and the (B) comprises an amino acid sequence selected from the group consisting of SEQ ID NOS: 17-20 or is a fragment of any one of SEQ ID NOS: 17-20 that comprises the fragment of SEQ ID NO:27.

11. The pharmaceutical composition of claim 1, wherein the total amount of (A) and (B) is greater than about 20% of total proteins in the composition.

12. The pharmaceutical composition of claim 1, wherein the total proteins do not include more than about 20% of myogenic antagonist factors, wherein the myogenic antagonist factors comprise IGFBP-3 (insulin like growth factor binding protein 3) and Dkk-3 (dickkopf WNT signaling pathway inhibitor 3).

13. The pharmaceutical composition of 12, wherein the myogenic antagonist factors further comprise Periostin, TGFb1, IL-6, FGF-21, TGFb2, TNFb, TGFb3, Activin A, TGFb1, and TGFa.

14. The pharmaceutical composition of claim 1, further comprising one or more pro-myogenic factors.

15. The pharmaceutical composition of claim 14, wherein the pro-myogenic factors comprise one or more of OPN, IGFBP-4, MMP-1, TSP-1, GROa, MMP-10, bIG-H3, hCGb, RGM-B, VEGF, Cripto-1, HGF, BMP-5, bFGF, IGF-2, PDGF-AA, FGF-19, and WISP-1.

16. The pharmaceutical composition of claim 1, wherein the total amount of pro-myogenic factors and the total amount of myogenic antagonist factors have a ratio of greater than 3:1.

17. A method for improving the muscle mass, strength, or function in a patient, comprising the step of:
administering to the patient a pharmaceutical composition comprising (A) a follistatin protein or a variant thereof, and (B) a fetuin A protein (alpha-2-HS-glycoprotein) or a fragment or variant thereof, wherein (B) is capable of binding to TGF-β, and wherein the total amount of (A) and (B) is greater than about 10% of total proteins in the composition.

18. The method of claim 17, wherein the administration is intramuscular, intravenous, or subcutaneous.

19. The method of claim 17, wherein the patient suffers from muscle injury, muscle atrophy, muscle dysfunction, or muscle fibrosis.

20. A method of preparing a secretome composition, comprising the steps of:
culturing stem cells in a protein-free media comprising trehalose at a concentration of from about 5% to 25% w/v;
harvesting supernatant from the sample and replacing the supernatant with a fresh protein-free media comprising trehalose at a concentration of from about 5% to 25% w/v; and
combining the harvested supernatant to prepare the secretome composition.

21. The method of claim 20, further comprising the step of reducing a volume of the combined harvested supernatant using a tangential flow filtration.

22. The method of claim 21, wherein the volume reduction by tangential flow filtration is 50:1 to 100:1.

23. The method of claim 22, wherein the tangential flow filtration utilizes a 1 to 5 kDa cutoff membrane.

24. The method of claim 20, further comprising the step of removing live cells from the harvested supernatant.

25. A method of preparing a secretome composition, the method comprising the steps of:
expanding pluripotent stem cells in culture medium comprising bFGF and Activin A for about 3 days to about 7 days;
exposing the pluripotent stem cells to a protein-free media comprising trehalose, wherein the trehalose concentration is between 5% and 25%;
harvesting the culture supernatant daily and replacing with additional protein-free media comprising trehalose for about 1 day to about 10 days;
combining the harvested culture supernatant to form a combined pool of culture supernatant;
homogenizing the combined pool of culture supernatant to form a homogenized combined pool of culture supernatant; and
filtering the homogenized combined pool of culture supernatant through a membrane filter to form a filtered homogenized combined pool.

26. The method of claim 25, further comprising the step of reducing a volume of the filtered homogenized combined pool using a tangential flow filtration.

27. The method of claim 26, wherein the volume reduction by tangential flow filtration is 50:1 to 100:1.

28. The method of claim 26, wherein the tangential flow filtration utilizes a 1 to 5 kDa cutoff membrane.

29. The method of claim 25, further comprising the step of removing live cells from the filtered homogenized combined pool.

30. A secretome composition comprising:
at least two myogenic agonists selected from the group consisting of Fetuin A, Follistatin, and Follistatin like-1,
wherein a combined concentration of the at least two myogenic agonists is between about 1 to about 20 pmol/mL.

31. The composition of claim 30, wherein the at least two myogenic agonists further comprise OPN, IGFBP-4, MMP-1, TSP-1, GROa (CXCL1), MMP-10, bIG-H3, hCGb, RGM-B, VEGF, Cripto-1, HGF, BMP-5, bFGF, IGF-2, PDGF-AA, FGF-19, WISP-1, or combinations thereof.

32. The composition of claim 30, wherein the composition further comprises at least two myogenic antagonists.

33. The composition of claim 32, wherein the composition has a concentration of the at least two myogenic antagonists below about 1 pmol/mL.

34. The composition of claim 32, wherein the at least two myogenic antagonists comprise Activin A and TGFb1.

35. The composition of claim 32, wherein a ratio of a concentration of the at least two myogenic agonists to the at least two myogenic antagonists is between about 4:1 and about 8:1.

36. The composition of claim 32, wherein a ratio of a concentration of the at least two myogenic agonists to the at least two myogenic antagonists is between about 5:1 and about 8:1.

37. The composition of claim 32, wherein a ratio of a concentration of the at least two myogenic agonists to the at least two myogenic antagonists is between about 5:1 and about 200:1.

38. A pharmaceutical composition, comprising:
at least two myogenic agonists selected from the group consisting of Fetuin A, Follistatin, and Follistatin like-1, wherein a combined concentration of the at least two myogenic agonists is between about 1 to about 20 pmol/mL;
a pharmaceutically acceptable carrier; and
a pharmaceutically acceptable preservative.

39. The composition of claim 38, wherein the pharmaceutically acceptable carrier is a physiological buffer with a pH of 7.0-7.8.

40. The composition of claim 38, wherein the pharmaceutically acceptable preservative is trehalose.

41. The composition of claim 38, wherein the pharmaceutical composition is a powder preserved by freeze-dry methods.

42. The composition of claim 38, wherein the pharmaceutical composition is sterilized by gamma irradiation.

43. The composition of claim 38, wherein the pharmaceutical composition is packaged in single dose glass container or ready to use single dose syringe.

44. The composition of claim 38, wherein the pharmaceutical composition further comprises a corticosteroid.

45. The composition of claim 38, wherein the pharmaceutical composition further comprises an anabolic steroid.

46. A method for treating muscles, the method comprising the step of:
administering to a subject a composition comprising at least two myogenic agonists selected from the group consisting of Fetuin A, Follistatin, and Follistatin like-1, wherein a combined concentration of the at least two myogenic agonists is between about 1 to about 20 pmol/mL; a pharmaceutically acceptable carrier; and a pharmaceutically acceptable preservative.

47. The method of claim 46, wherein the composition is administered by injection.

48. The method of claim 47, wherein the injection is administered intramuscularly, intravenously, or subcutaneously.

49. The method of claim 47, wherein the composition is administered once, daily, weekly, or monthly.
